(19) United States
(12) Patent Application Publication
(10) Pub. No.: US 2004/0175764 A1
(43) Pub. Date: Sep. 9, 2004

(54) Image processing apparatus, image processing program, recording medium, and image processing method

(76) Inventors: Hiroto Nishiyama, Tokyo (JP); Koutatsu Oura, Shanghai (CN)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/725,791

(22) Filed: Dec. 2, 2003

(30) Foreign Application Priority Data
Jan. 6, 2003 (JP) 2003-000615
Jan. 6, 2003 (JP) 2003-000616
Jan. 6, 2003 (JP) 2003-000617

(51) Int. Cl. G01N 33/53; G01N 33/567

(52) U.S. Cl. 435/7.2

(57) ABSTRACT

An image processing apparatus realized by running an image processing program on a PC. In the image processing apparatus, a plurality of thumbnail images included in images stored in the PC are displayed in an array in a browse mode; a classification check box is displayed in each of the images displayed in the browse mode in order to put the corresponding image into any of categories, when one or more categories are designated among the categories; a plurality of images each of which belongs to any of the designated categories are displayed in a virtual light box mode to compare the images; and at least one image is selected and designated from among the images displayed in the virtual light box mode with selective collection.

### CROSS REFERENCE TO RELATED APPLICATION(S)

This application claims benefit of Japanese Application Nos. 2003-615, 2003-616, 2003-617 filed in Japan on Jan. 6, 2003, the contents of which are incorporated by this reference.

### BACKGROUND OF THE INVENTION

1. Field of the Invention

The present invention relates to an image processing apparatus, an image processing program, a recording medium, and an image processing method for displaying several images of a plurality of images on the same screen to make a comparison therebetween in order to select a desired image.

2. Description of Related Art

As the recent Internet technology advances, the demand for posting pictures on a website is increasing. Accordingly, digital cameras capable of easily capturing digital still images are finding widespread use. The digital camera is establishing a firm place in not only the above-mentioned private use but also professional use such as news photos and commercial photos because no film exposure and development is required, an image can be transmitted to a newspaper office via communication, the development of image pickup devices with a large number of pixels is realizing high-definition images which are favorably compared with images captured by film cameras, and a captured image can be easily subjected to digital processing.

As contrasted with the film camera in which, after an image is recorded, the recorded image cannot be rerecorded, the digital camera uses a recording medium such as a memory card in which an image can be repetitively recorded. Advantageously, the user can more freely capture an image using a digital camera without being afraid of failed shots. In addition, as recent recording media increase in capacity, the number of images managed by the user is trending toward enormous volumes.

As the number of images increases, it is harder for the user to handle many images. In other words, when a private user selects at least one image for, for example, a new year greeting card from among many images, or when a professional photographer selects at least one image to be printed in a magazine from among many images, it is very troublesome to select one image.

On the basis of the above-mentioned viewpoint, various image processing techniques have been proposed for easily selecting desired images from among many images and grouping the selected images.

Among the techniques, for example, Japanese Unexamined Patent Application Publication No. 2001-109080 discloses an image creating apparatus for creating an index image constituting one single image which comprises a plurality of images arranged in a matrix form, the apparatus comprising: means for inputting an image together with added information; means for designating a classification item which is used when the input image is put into a group; means for putting the input image into a group in accordance with the designated classification item with reference to the added information of the image; and means for arranging image groups so that each group is arranged in an array and further for editing an index image with visual information so that the kinds of image groups can be recognized.

According to Japanese Unexamined Patent Application Publication No. 2001-109080, the input images are divided into said groups on the basis of the added information. If an image has no added information or added information is not proper, images cannot always be classified as intended.

Japanese Unexamined Patent Application Publication No. 9-258709 discloses a split screen display method in an image communication terminal. The image communication terminal comprises display means for converting a signal received from a communication path through a communication control unit, and for displaying an image, the image being obtained through a display control unit; split screen display means for dividing a screen into areas to simultaneously display a plurality of images when a plurality of image information items are received; and storage means for storing received images. According to the method, a specified image selected from among images displayed through the split screen display means is left, and other images take the place of image information items, which are stored in the terminal and are not displayed yet, and the images are displayed.

According to the method disclosed in Japanese Unexamined Patent Application Publication No. 9-258709, a user cannot view candidate images to be compared until the images are displayed on a split screen. The efficiency of comparison is not necessarily good.

In selecting an image from among images captured by a digital camera as mentioned above, a plurality of images are placed and are compared side-by-side in a manner similar to a procedure for selecting a photo from among photos taken through a film camera or for selecting a film from among films taken by the film camera.

As an example of techniques for making such a comparison, Japanese Unexamined Patent Application Publication No. 11-45334 discloses an image comparative display method comprising: a step of displaying a plurality of images on a display device based on a plurality of image data items corresponding to the images; a step of selecting one image as a reference image from among the images; a step of setting a processing parameter for performing predetermined image processing to the reference image; a step of performing the predetermined image processing to the reference image based on the processing parameter and displaying the resultant image in the display device; and a step of performing the predetermined image processing to the images excluding the reference image based on the processing parameter and displaying the resultant images in the display device.

The plurality of images placed and displayed are subjected to processing such as zoom-in, zoom-out, target-area-shifting, field extraction, or rotation, the images being operatively interlocked with each other. While the selected images are processed, the images are compared with each other, thus increasing the efficiency of operation.

Even if images are processed, for example, zoomed in or zoomed out and/or moved such that they are operatively interlocked with each other as disclosed in Japanese Unexamined Patent Application Publication No. 11-45334, the images are not always easily compared with each other. In other words, in comparison between various images, for example, the images may be different from each other in the number of pixels (resolution) constituting each image, vertically oriented images and horizontally oriented images may be mixed, or the images may be captured at various zoom magnifications of a photographing optical system. When the above-mentioned various images are merely placed side-by-side and displayed, main subjects of the respective images are generally different from each other in display size. For example, even if the images are zoomed in such that they are operatively interlocked with each other, images cannot always be easily compared with each other because the main subjects are merely enlarged while the display sizes of the main subjects are different from each other.

### SUMMARY OF THE INVENTION

It is a first object of the present invention to provide an image processing apparatus, an image processing program, a recording medium, and an image processing method capable of selecting a desired image from among many images employing a simple operation.

It is a second object of the present invention to provide an image processing apparatus, an image processing program, a recording medium, and an image processing method capable of comparing with efficiency several images selected among a large number of images to select a desired image.

It is a third object of the present invention to provide an image processing apparatus, an image processing program, a recording medium, and an image processing method capable of comparing a plurality of images with good operational ease so that a comparison therebetween is easily made.

In brief, the present invention provides an image processing apparatus including: first display means for displaying one or more images of a plurality of images; classification means for putting each image displayed through the first display means into any one of a plurality of categories; designation means for designating one or more categories among the categories; second display means for displaying one or more images each of which belongs to any of the one or more categories designated by the designation means; and selective designation means for selecting and designating an image among the images displayed through the second display means.

Also, in brief, the present invention provides an image processing apparatus including display means, selection means, display control means, display holding means, target image-space shifting means, and selective designation means. The display means displays first, second, and third display areas in the same screen. The first display area is used to display a plurality of reduced images arranged in a first predetermined order. The second display area includes two or more predetermined number of image display spaces arranged in a second predetermined order, one of the image display spaces being selectively moved as a target image display space, the second display area being used to compare a plurality of images. The third display area is used to store an image selected from the images displayed in the second display area and display the selected image as a reduced image. The selection means selects one reduced image from among the reduced images displayed in the first display area. The display control means controls the display means to display an image, corresponding to the reduced image selected through the selection means, in the target image display space in the second display area. The display holding means holds the display state of the image displayed in the target image display space in the second display area such that the display state is releasable. The target image-space shifting means automatically moves the target image display space by one in accordance with the second order when the display holding means holds the image. The selective designation means selects and designates the image displayed in the second display area to store the image into the third display area.

Further, in brief, the present invention provides an image processing apparatus including display control means, comparison-image-size adjusting means, image processing means, and interlocking means. The display control means controls a plurality of different images so that the images are displayed in respective same-sized image display areas in the same screen. The comparison-image-size adjusting means enlarges or reduces the display size of at least one of the images so that subjects included in the respective images displayed in the image display areas are displayed in substantially the same size on the screen, the subjects indicating the same subject. The image processing means performs image processing to any one or more images of the images displayed in the image display areas. The interlocking means allows the image processing means to perform image processing to all of the images displayed in the image display areas such that the images are operatively interlocked with each other.

The above and other objects, features and advantages of the invention will become more clearly understood from the following description referring to the accompanying drawings.

### BRIEF DESCRIPTION OF THE DRAWING(S)

FIG. 1 is a state transition diagram showing a state transition in selecting an image according to a first embodiment of the present invention;

FIG. 2 is a block diagram schematically showing the structure of a personal computer (PC) in the first embodiment;

FIG. 3 is a flowchart showing the operation which is performed mainly through classification means according to the first embodiment;

FIG. 4 is a flowchart showing the operation which is performed mainly through data-narrowing display means according to the first embodiment;

FIG. 5 is a flowchart showing the operation in a browse mode, the operation being performed mainly through comparative selection means according to the first embodiment;

FIG. 6 is a flowchart showing the operation in a view mode or a full-screen display, the operation being performed mainly through the comparative selection means according to the first embodiment;

FIG. 7 is a flowchart showing the operation in a virtual light box (VLB) mode, the operation being performed mainly through the comparative selection means according to the first embodiment;

FIG. 8 is a diagram showing an example of a screen display when a collection area is reduced and displayed in the browse mode according to the first embodiment;

FIG. 9 is a diagram showing an example of the screen display when the collection area is displayed as a regular window in the browse mode according to the first embodiment;

FIG. 10 is a diagram showing an example of the screen display in the view mode according to the first embodiment;

FIG. 11 is a diagram showing an example of a display in a two-image-vertically-split pattern in the view mode according to the first embodiment;

FIG. 12 is a diagram showing an example of a display in a four-image-split pattern in the view mode according to the first embodiment;

FIG. 13 is a block diagram schematically showing the structure of a PC according to a second embodiment of the present invention;

FIG. 14 is a flowchart showing the operation which is performed mainly through classification means according to the second embodiment;

FIG. 15 is a flowchart showing the operation which is performed mainly through data-narrowing display means according to the second embodiment;

FIG. 16 is a flowchart showing the operation in the browse mode, the operation being performed mainly through comparative selection means according to the second embodiment;

FIG. 17 is a flowchart showing the operation in the view mode or full-screen display, the operation being performed mainly through the comparative selection means according to the second embodiment;

FIG. 18 is a flowchart showing the operation in the VLB mode, the operation being performed mainly through the comparative selection means according to the second embodiment;

FIGS. 19A to 19E are diagrams showing the operation for adding an image from a thumbnail area to a comparison area in the VLB mode of an image processing apparatus according to the second embodiment when a collective-add-to-list check box is OFF and a selective-add-to-list check box is ON;

FIGS. 20A to 20E are diagrams showing the operation for adding images from the thumbnail area to the comparison area in the view mode of the image processing apparatus according to the second embodiment when the collective-add-to-list check box is ON and the selective-add-to-list check box is ON;

FIGS. 21A to 21D are diagrams showing the operation for adding an image from the thumbnail area to the comparison area in the view mode of the image processing apparatus according to the second embodiment when the collective-add-to-list check box is OFF and the selective-add-to-list check box is OFF;

FIGS. 22A to 22D are diagrams showing the operation for adding images from the thumbnail area to the comparison area in the view mode of the image processing apparatus according to the second embodiment when the collective-add-to-list check box is ON and the selective-add-to-list check box is OFF;

FIGS. 23A to 23E are diagrams showing the operation for adding an image from the thumbnail area to the comparison area in the view mode of the image processing apparatus according to the second embodiment when the collective-add-to-list check box is OFF, the add operation being effected by an image add button;

FIGS. 24A to 24E are diagrams showing the operation for adding images from the thumbnail area to the comparison area in the view mode of the image processing apparatus according to the second embodiment when the collective-add-to-list check box is ON, the operation being effected by the image add button;

FIG. 25 is a block diagram schematically showing the structure of a PC according to a third embodiment of the present invention;

FIG. 26 is a flowchart showing the operation which is performed mainly through classification means according to the third embodiment;

FIG. 27 is a flowchart showing the operation which is performed mainly through data-narrowing display means according to the third embodiment;

FIG. 28 is a flowchart showing the operation in the browse mode, the operation being performed mainly through comparative selection means according to the third embodiment;

FIG. 29 is a flowchart showing the operation in the view mode or full-screen display, the operation being performed mainly through the comparative selection means according to the third embodiment;

FIG. 30 is a flowchart showing the operation in the VLB mode, the operation being performed mainly through the comparative selection means according to the third embodiment;

FIGS. 31A to 31F are diagrams showing a process of facilitating comparing images of which resolutions only are different, according to the third embodiment;

FIGS. 32A to 32F are diagrams showing a process of facilitating comparing images of which orientations only are different, according to the third embodiment; and

FIGS. 33A to 33D are diagrams showing a process of facilitating comparing images of which only zoom magnifications are different, according to the third embodiment.

### DETAILED DESCRIPTION OF THE PREFERRED EMBODIMENT(S)

Embodiments of the present invention will now be described hereinbelow with reference to the drawings.

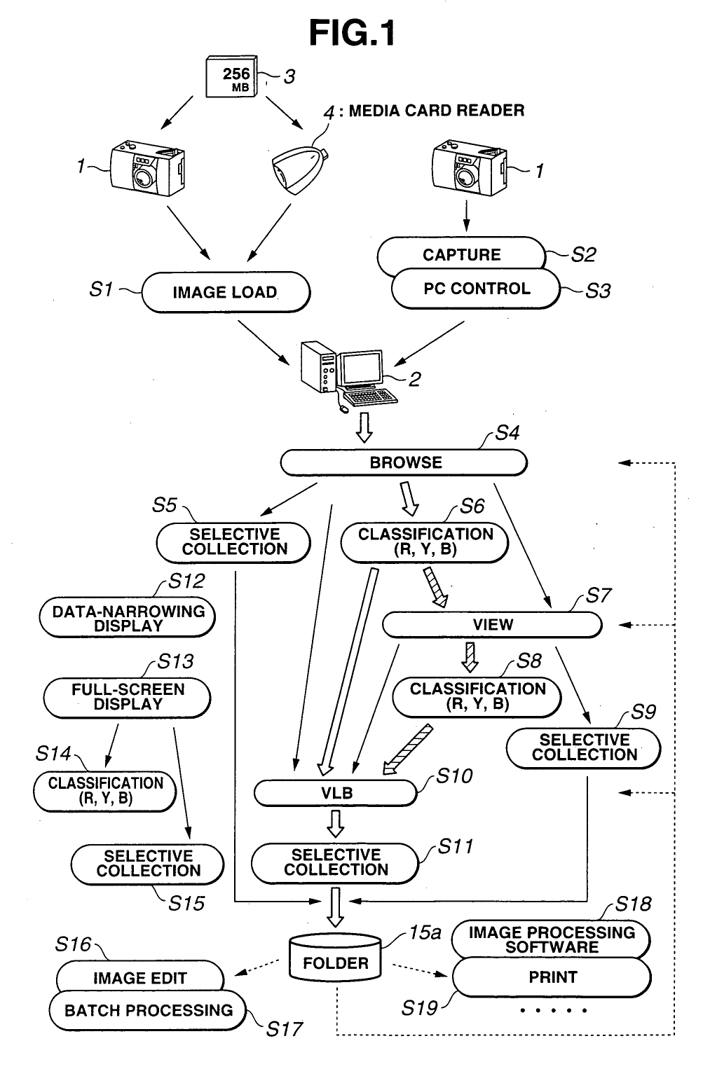

FIGS. 1 to 12 show a first embodiment of the present invention. FIG. 1 is a state transition diagram showing a state transition in image selection.

According to the first embodiment, an image processing program runs on a personal computer (PC) 2 to allow the PC 2 to function as an image processing apparatus for selecting a desired image from among a plurality of images.

A digital camera 1 loads, for example, a media card 3 constituting a card-shaped recording medium and records a captured image in the media card 3. The captured image recorded in the media card 3 is transferred to the PC 2 via the media card reader 4 or by connecting the digital camera 1 directly to the PC 2, and the image is loaded into the PC 2 (S1).

The PC 2 is connected to the digital camera 1 through a two-way communication line such as IEEE 1394 or USB 2.0 port serving as means for transferring a captured image from the digital camera 1 to the PC 2. The PC 2 can control the digital camera 1 to capture an image and load the captured image into the PC 2 (S2, S3).

An image loaded in the PC 2 is stored every shooting. Many images may be recorded in the PC 2 over time.

As mentioned above, when an image is used for a magazine, a web site on the Internet, or a new year greeting card, an image appropriate for the purpose is selected from among the many images and the selected image is subjected to image processing when necessary.

In the selection, the image processing program stored in the PC 2 is run on an operating system for controlling the fundamental operation of the PC 2.

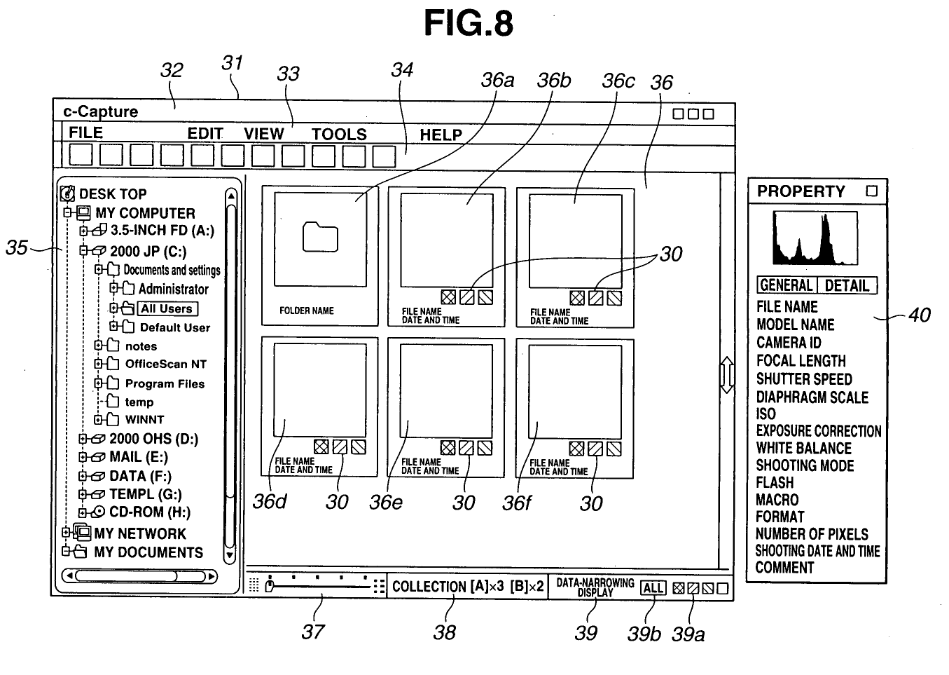

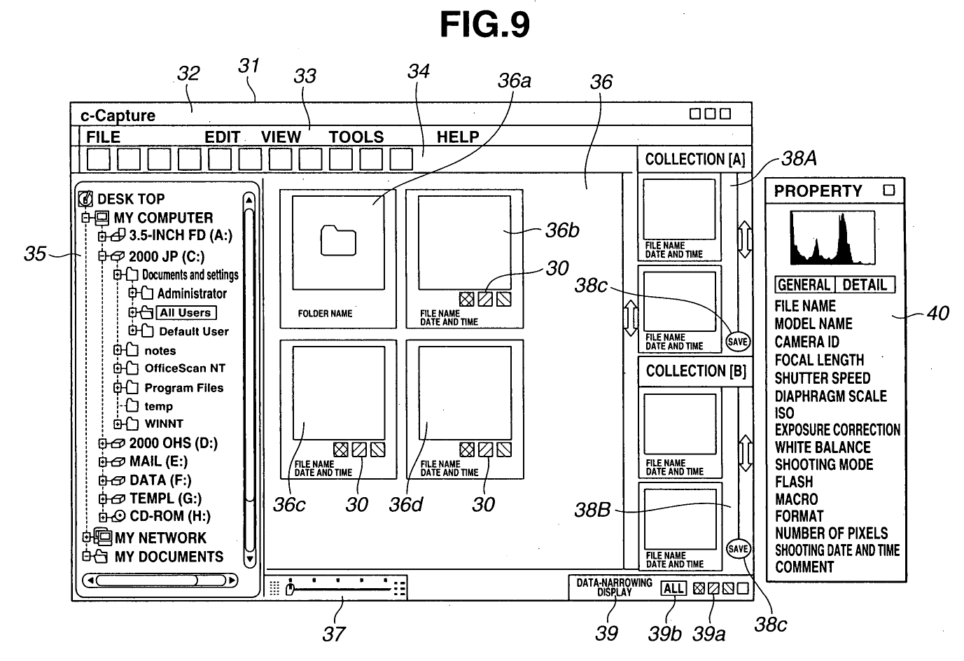

After the completion of various processes for initialization, a window (refer to FIGS. 8 and 9) in a browse mode is displayed (S4). The browse mode will be described later.

In the browse mode, a plurality of reduced images (thumbnail images) are displayed in an array. An image is selected from among the images and the selected image is moved to a collection field by dragging and dropping or the like, resulting in performing a selective collection (S5).

As mentioned above, however, the selection of a target image from among many images requires a user's power of memory, attention, and labor. Therefore, images are classified into categories (S6). The user can select an image from among the classified images.

As an example of the classification, three categories such as high priority, middle priority, and low priority are provided. Images which are not included in those categories are the lowest priority images. Therefore, images are classified into four categories. After the completion of classification, the user designates a certain category. Consequently, only images included in the designated category can be displayed for the user to browse.

According to the first embodiment, colored check boxes, for example, red (R), yellow (Y), and blue (B) check boxes are provided. The user checks any one of the boxes. As for the categories, in addition to the classification in priority, images can be classified in accordance with shooting locations, namely, studios, indoors, and outdoors. Images can also be classified in accordance with time zones, namely, day shooting, morning or evening shooting, and night shooting. Classification is not limited to the above examples. The user can freely use categories depending on the user's purpose.

In this instance, the classification of the check boxes is not limited to color. For example, the check boxes can be classified using figures such as □, O, and [symbol] . Other means which can be easily recognized can be generally used.

In this manner, images are classified into categories in the browse mode or another mode in the PC 2. After that, an image can be selected using a VLB (virtual light box), which will be described later.

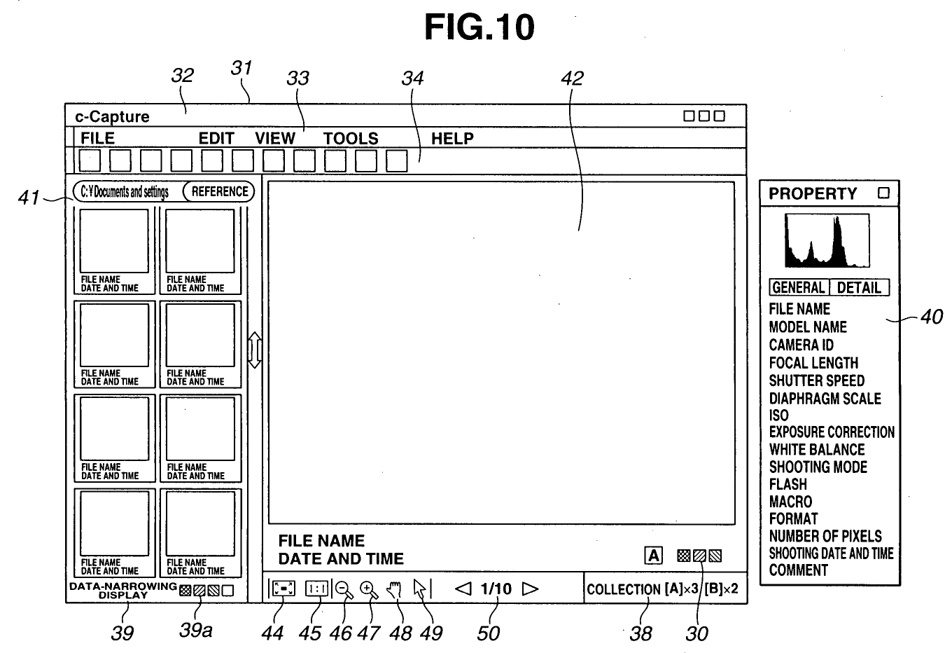

In the browse mode in S4, the classification is performed in S6. After that, the mode can be changed to a view mode (refer to FIG. 10). Alternatively, the browse mode in S4 can be directly shifted to the view mode.

In the view mode, one single image selected in a thumbnail area 41 (refer to FIG. 10) is enlarged and is displayed in a view area 42 (refer to FIG. 10) (S7). In the view mode, the user can classify images (S8) or performing selective collection on images (S9) while viewing the enlarged image.

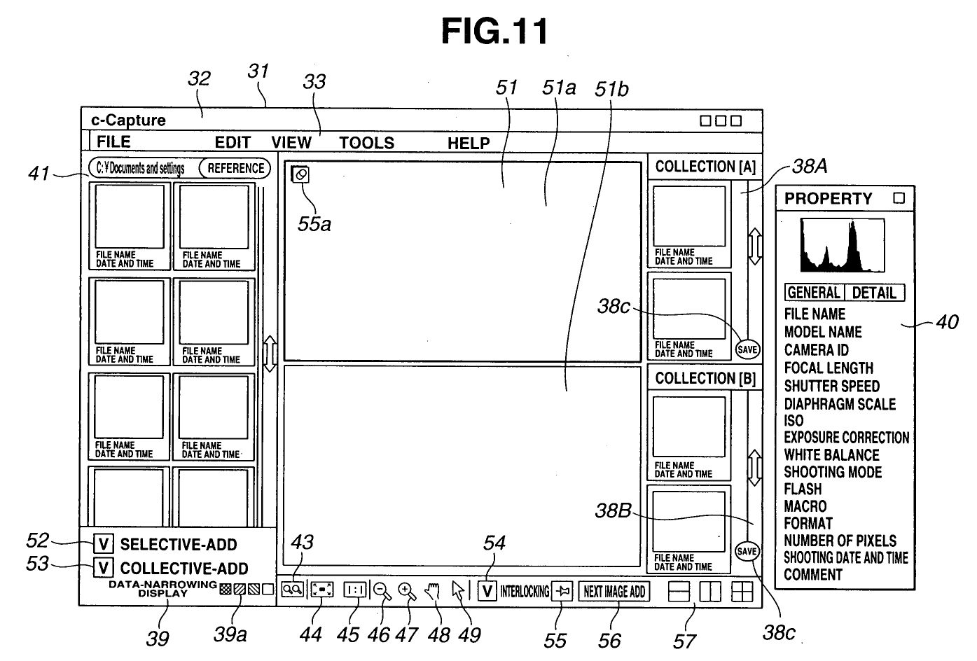

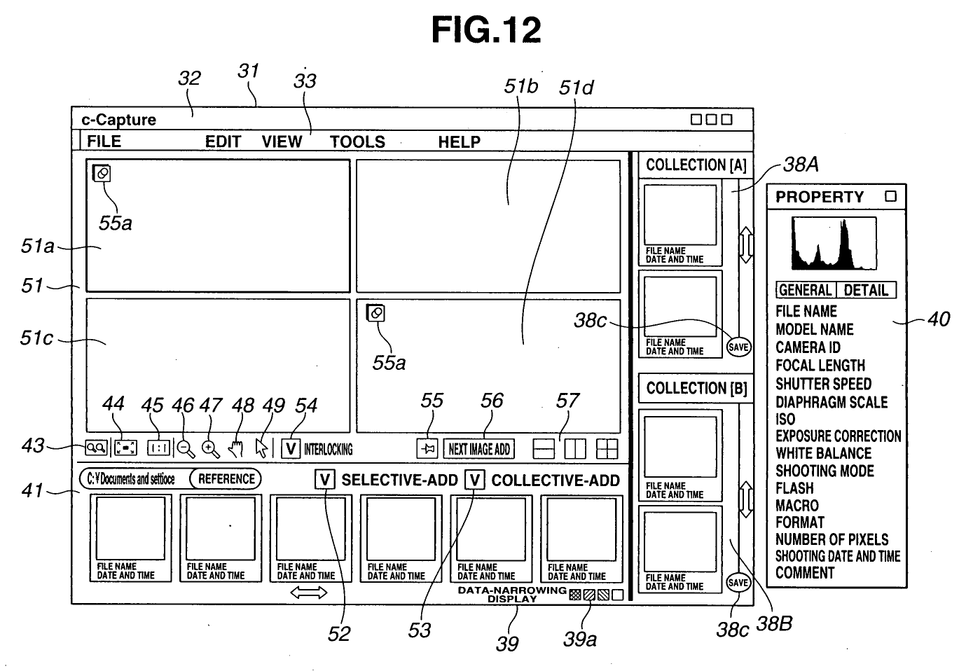

The browse mode in S4 can be shifted to a VLB mode (refer to FIGS. 11 and 12) in either case of the following: Directly from the browse mode in S4, after the completion of classification in S6, which is done directly from the browse mode in S4, directly from the view mode in S7, or after the completion of classification in S8, which is done directly from the view mode in S7.

In the VLB mode, a comparison between two or four images, namely, a relatively small number of images is made for selection (S10). The VLB mode, specifically, is used to determine an image to be selected from a plurality of images obtained by shooting the same scene with varied exposure, alternatively, a shot to be selected from among similar shots, the VLB mode is often used.

In the VLB mode, therefore, selective collection can be performed (S11).

Images displayed in a browse area 36 (refer to FIGS. 8 and 9) in the browse mode in S4, those displayed in the thumbnail area 41 (refer to FIG. 10) in the view mode in S7, and those displayed in the thumbnail area 41 (refer to FIGS. 11 and 12) in the VLB mode in S10 can be subjected to data-narrowing (S12). More specifically, one or more categories are designated among the categories classified as mentioned above, so that only images belonging to the designated categories are narrowed to and are displayed. Images which are not included in any category can also be narrowed to and be displayed. Consequently, as compared with the selection of an image from among all images, the selection operation can be performed easier.

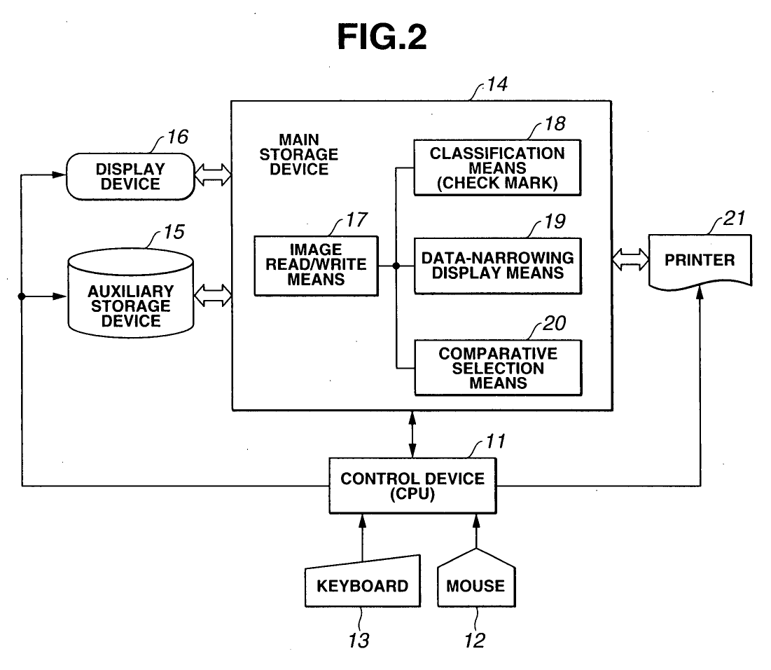

In each of the browse mode in S4, the view mode in S7, and the VLB mode in S10, after one image is selected, the mode can be changed to a full-screen display mode (S13). In the view mode in S7, one single image is displayed only in the view area 42 in a display window according to the image processing program, the display window being shown in the whole screen or one part of the screen. Contrarily, in the full-screen mode, one single image is displayed in the whole of a display area on a display device 16 (FIG. 2). The full-screen display mode differs from the view mode in this respect.

In the full-screen display mode, the classification (S14) and the selective collection (S15) can be performed. However, since an image fills the screen, the classification check box and a selective collection display are superimposed on the image and are operably displayed.

Images, each of which is selected in any of S5, S9, S11, and S15 mentioned above, can be stored (copied) in a lump into a folder 15a (an automatically-set folder may be used or a folder, which the user manually selects or creates, may also be used) in an auxiliary storage device 15 (refer to FIG. 2). The auxiliary storage device 15 will be described later.

After that, an image file, which is stored in the folder 15a as the same area in a recording medium, is subjected to image edit (S16) such as low-pass filtering and sharpness compensation serving as functions included in the image processing program. Alternatively, the image file is subjected to batch processing (S17) based on a batch file described so that a plurality of functions can be performed in a lump.

The image file stored in the folder 15a can be processed (S18) according to image processing software different from the image processing program. The image file can also be printed through a printer 21 (refer to FIG. 2) (S19). The printer 21 will be described later.

Further, the image stored in the folder 15a can be again read in the browse mode in S4, the view mode in S7, or the VLB mode in S10 and be repetitively subjected to the above-mentioned processing.

Although it is not shown in detail in FIG. 1, the browse mode in S4, the view mode in S7, and the VLB mode in S10 can be shifted to each other bidirectionally. Further, each of the above modes can be shifted to the full-screen display mode in S13 and the mode can also be returned to the preceding mode.

Although it is not shown in FIG. 1, images displayed in a comparison area 51 (refer to FIGS. 11 and 12) in the VLB mode may be classified into categories.

FIG. 2 is a block diagram schematically showing the structure of the PC 2.

The PC 2 comprises: a control unit 11 comprising a CPU etc. and functioning as storage control means; a mouse 12 for pointing a position on the screen of the display device 16 in order to perform the operation, the display device 16 being described later; a keyboard 13 used for character input; a main storage device 14 comprising a RAM and serving as a work area of the control unit 11; the auxiliary storage device 15 comprising a readable recording medium such as a hard disk in which a hierarchical file system having folders is constructed, and the image processing program and image data are recorded; and the display device 16 for displaying in a visual form window images and other various information items according to the image processing program.

The PC 2 connects to, for example, the printer 21. Under the control of the control unit 11, image data transferred from the main storage device 14 can be printed through the printer 21.

In the main storage device 14, an operating system for controlling the fundamental operation of the PC 2 and the image processing program running on the operating system are loaded such that the system and the program can be executed. In addition, various data items such as image data are loaded such that each data is processed.

The image processing program comprises: image read/write means 17 constituting storage control means for reading image data from the auxiliary storage device 15, writing processed image data into the auxiliary storage device 15, and/or outputting the processed image data to the printer 21; classification means 18 for putting an image, stored in a predetermined folder in the auxiliary storage device 15, into any one of a plurality of preset categories; data-narrowing display means 19 for displaying only images belonging to at least one designated category in a predetermined display area in the display device 16 when one or more categories are designated among the categories of images classified through the classification means 18; and comparative selection means 20 for displaying two or more images read from the auxiliary storage device 15 through the image read/write means 17 in a predetermined display area in the display device 16 to make a comparison and selection therebetween.

Before describing the operation according to the image processing program, examples of screen displays according to the image processing program will now be described with reference to FIGS. 8 to 12.

FIG. 8 is a diagram showing one example of a screen display image when the collection area is reduced and displayed in the browse mode.

A window 31 according to the image processing program includes: a title bar 32 where the name of the image processing program is displayed; a menu bar 33 where various operations related to the image processing program are performed using pull-down menu items; a tool bar 34 where various operating functions related to the image processing program are displayed as icons and each icon is pointed and operated using the mouse 2 or the like; a folder tree 35 displaying drives and folders in the auxiliary storage device 15 of the PC 2 in a tree structure (directory structure), the folder tree 35 being constructed such that when the user double-clicks on a folder including images, the predetermined number of images stored in the folder are displayed in a browse area 36, which will be described later; the browse area 36 constituting browse display means for displaying an array of reduced images 36b to 36f together with respective file names and shooting dates and times and a folder 36a in which these images are stored, and also displaying classification check boxes 30, serving as classification means used to designate categories of the images 36b to 36f, so that any one of alternative segments of each check box 30 can be checked; a display-size change slider 37 for changing the size of each image displayed in the browse area 36 (properly changing the number of displayed images depending on the changed image size); a collection field 38 constituting selective designation means serving as an area where an image selected from the images displayed in the browse area 36 is entered (in this example, two collection areas A and B are provided for selective designation); and a classification check mark tool box 39 constituting designation means comprising check boxes 39a and 39b used to designate at least one category of the images displayed in the browse area 36, the check box 39a comprising segments (three hatched segments in the diagram) corresponding to the categories which can be simultaneously selected (simultaneous multiple selection can be performed in contrast to the classification check box 30) and a segment (a blank segment in the diagram) indicating that displayed images do not belong to any category, the check box 39b indicating that all of images (ALL) are displayed irrespective of the categories. A property window 40 is further displayed as a window separated from the window 31. The property window 40 shows the properties of an image.

In the property window 40, as one of the properties of an image, a brightness histogram generated from the image is displayed. Further, as general properties, a file name, a model name, a camera ID, a focal length, a shutter speed, an diaphragm scale, ISO, exposure correction, white balance, shooting mode, flash, macro, a format, the number of pixels, shooting data and time, a comment, and the like can be displayed. The property window 40 can further display advanced properties in addition to the above general properties.

FIG. 9 is a diagram showing one example of a screen display image when the collection areas are displayed as regular-sized areas in the browse mode.

The collection field 38 is enlarged such that the collection areas constituting the field 38 are displayed as regular-sized areas on the right of the browse area 36. Reference numeral 38A denotes the collection area A and reference numeral 38B denotes the collection area B. The reduced images which have been selectively entered are displayed (as thumbnail images) in the respective collection areas 38A and 38B.

Further, a save button 38c is displayed in each of the collection areas 38A and 38B. The save button 38c is used to actually store (copy) the selected image group into the folder 15a (refer to FIG. 1) in the auxiliary storage device 15.

FIG. 10 is a diagram showing an example of a screen display image in the view mode.

The title bar 32, menu bar 33, and tool bar 34 are displayed. Further, the thumbnail area 41, where an array of thumbnail images is displayed, is displayed in a left portion in the window 31 such that image display can be set using the classification check mark tool box 39.

In most portions from the central toward right in the window 31, the view area 42 serving as view display means is displayed such that any category can be designated using the classification check box 30.

Below the view area 42 in the window 31, further, a fit button 44, a 1× magnification button 45, a zoom-out button 46, a zoom-in button 47, a moving-pointer set button 48, a normal-pointer set button 49, a next-or-preceding image button 50, and the reduced collection field 38 are displayed. The fit button 44 is used to adjust the size of an image such that the image is displayed in the view area 42 as large as possible without having a part lying off-screen. The 1× magnification button 45 is indicated as “1:1” and is used to display an image at 1× magnification such that pixels constituting the image correspond to display pixels of the display device 16 in a one-to-one relationship. The zoom-out button 46 is used to zoom out an image displayed in the view area 42. Each time the user clicks on the zoom-out button 46, the image is zoomed out by a predetermined amount. The zoom-in button 47 is used to zoom in an image displayed in the view area 42. Each time the user clicks on the zoom-in button 47, the image is zoomed in by a predetermined amount. When the user clicks on the moving-pointer set button 48, a pointer responding to the operation of the mouse 12 is set to a moving pointer for moving an image. The user drags the mouse while pressing a click button of the mouse on the moving-pointer set button 48, thus moving an image. The normal pointer is shown by, for example, an arrow icon. The moving pointer is shown by, for example, a hand-shaped icon. The normal-pointer set button 49 is used to reset the moving pointer to the normal pointer. The next-or-preceding image button 50 is used to display the next image or the first preceding image in the view area 42 in accordance with the arranged order in the thumbnail area 41.

FIG. 11 is a diagram showing an example of a two-image-vertically-split pattern display in the VLB mode.

In the window 31 in a two-image-vertically-split pattern, the title bar 32, menu bar 33, and thumbnail area 41 are displayed. Further, the comparison area 51 is displayed. The comparison area 51 constitutes comparative display means for displaying selected images in the thumbnail area 41 in order to compare the images. Two display spaces 51a and 51b are vertically arranged in the comparison area 51.

Below the thumbnail area 41, the classification check mark tool box 39, a selective-add-to-list check box 52, and a collective-add-to-list check box 53 are displayed together. To add an image in the thumbnail area 41 to the comparison area 51, a thumbnail image displayed in the thumbnail area 41 is usually added to the comparison area 51 via dragging and dropping. In contrast to the usual add, when the selective-add-to-list check box 52 is checked, each time the user selects an arbitrary thumbnail image displayed in the thumbnail area 41 with a single click of the mouse 12, he or she can add the image to the comparison area 51. When the collective-add-to-list check box 53 is checked, the user merely drags and drops (or selects in the case where the selective-add-to-list check box 52 is checked) one of the thumbnail images displayed in the thumbnail area 41, so that a plurality of images in the thumbnail area 41 can be added to the comparison area 51 in a collective manner in accordance with the arranged order.

Below the comparison area 51, the fit button 44, 1× magnification button 45, zoom-out button 46, zoom-in button 47, moving-pointer set button 48, and normal-pointer set button 49 are displayed. Further, a compare button 43, an interlocking- operation check box 54, a pushpin button 55, and an image add button 56 are displayed. The compare button 43 is used to set the sizes of respective images displayed in the comparison area 51 such that the images can be easily compared to each other. When the interlocking-operation check box 54 is checked, respective images displayed in the comparison area 51 are zoomed out or zoomed in using the zoom-out button 46 or the zoom-in button 47 such that the images are operatively interlocked with each other, and/or when the moving pointer is set using the moving- pointer set button 48, the moving process is performed such that the images are operatively interlocked with each other. The pushpin button 55 is used to temporarily fasten an active image displayed in one space of the comparison area 51 so that the image is not replaced by another one. When the user clicks on the image add button 56, the next image (image group) in the array displayed in the thumbnail area 41 is added to a space where an image is not temporarily fastened by the pushpin button 55. In the display example of FIG. 11, a pushpin mark 55a indicating temporary fastening by the pushpin button 55 is displayed on an image displayed in the display space 51a of the comparison area 51.

Below the collection areas 38A and 38B, a display-mode change button 57 is displayed. The display-mode change button 57 is used to change the layout of the comparison area 51 to any one of a two-image-vertically-split pattern, a two-image-horizontally-split pattern, and a four-image-split pattern.

FIG. 12 is a diagram showing an example of a four-split pattern display in the VLB mode.

In the four-image-split pattern display, the comparison area 51 is displayed in portions from the central toward left in the window 31. The comparison area 51 includes four display spaces 51a, 51b, 51c, and 51d in an array of 2×2. The spaces 51a, 51b, 51c, and 51d are arranged at the upper-left, upper-right, lower-left, and lower-right positions in that order.

Below the comparison area 51, the thumbnail area 41 is displayed. In an upper portion of the thumbnail area 41, the selective-add-to-list check box 52 and collective-add-to-list check box 53 are displayed. In a lower-right portion of the thumbnail area 41, the classification check mark tool box 39 is displayed.

In a portion between the comparison area 51 and the thumbnail area 41, the compare button 43, the fit button 44, the 1× magnification button 45, the zoom-out button 46, the zoom-in button 47, the moving-pointer set button 48, the normal-pointer set button 49, the interlocking-operation check box 54, the pushpin button 55, the image add button 56, and the display-mode change button 57 are arranged and displayed.

Further, at the right of the comparison area 51 and the thumbnail area 41, the collection areas 38A and 38B are displayed in a manner similar to FIG. 11.

In the display example of FIG. 12, the pushpin marks 55a each indicating that the corresponding image is temporarily fastened by the pushpin button 55 are displayed at the images displayed in the display spaces 51a and 51d of the comparison area 51.

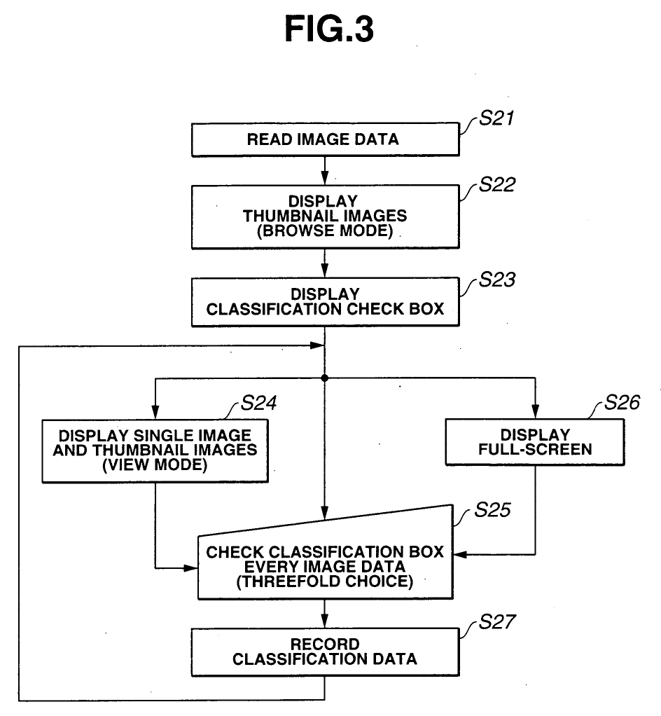

FIG. 3 is a flowchart showing the operation which is performed mainly through the classification means 18 (FIG. 2).

At the start of a process, image data is read from the auxiliary storage device 15 through the image read/write means 17 (step S21). In the browse mode, the folder 36a and the thumbnail images 36b, 36c, . . . are displayed in the browse area 36 (step S22). When the thumbnail images 36b, 36c, . . . are recorded at the respective headers of the corresponding image files, the thumbnail images are read and used. When a certain image file does not include any thumbnail image, a thumbnail image is generated from the corresponding image data and is then used.

Subsequently, the classification check box 30 (refer to FIGS. 8 and 9) is displayed in each of the thumbnail images 36b, 36c, . . . (step S23). As mentioned above, the user alternatively selects any of the segments constituting the classification check box 30 to designate a category to which the corresponding image belongs.

When the browse mode is shifted to the view mode, the thumbnail images are displayed in the thumbnail area 41 and an image designated among the thumbnail images is displayed in the view area 42 (if any image is not designated, the first image displayed in the thumbnail area 41 is displayed in the view area 42) (step S24). As shown in FIG. 10, the classification check box 30 is displayed also in the view area 42.

When any of the thumbnail images 36b, 36c, . . . is designated such that the corresponding image is displayed in the full screen, the image is displayed in the full screen (step S26). In the full screen display (not shown), the classification check box 30 is displayed as mentioned above.

Any one of the segments constituting the classification check box 30 is checked in a threefold-choice manner for every image data in any of the browse mode in step S23, the view mode in step S24, and the full screen display in step S26 (step S25). Classification data formed in the above step is stored as, for example, an exclusive classification file independent of the image file in the auxiliary storage device 15 (step S27). The operation for storing classification data is performed every image. Each time the classification check box 30 is checked, the storage operation is performed.

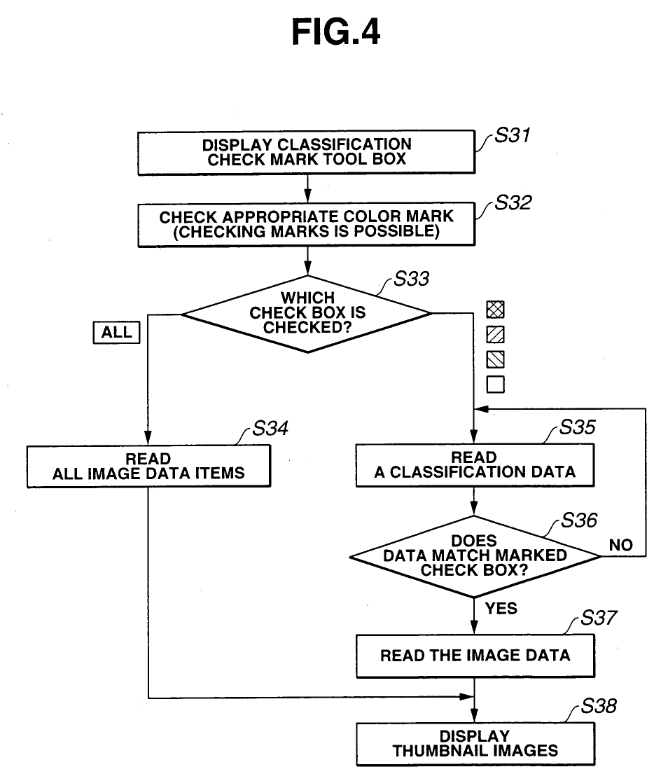

FIG. 4 is a flowchart showing the operation which is mainly performed through the data-narrowing display means 19.

At the start of a process, the classification check mark tool box 39 is displayed (refer to FIGS. 8 to 12) (step S31).

When necessary, the user checks either box of the following: the check box 39a for indicating any one or more segments (a plurality of segments can be simultaneously checked as mentioned above) or for indicating the segment that the corresponding image does not belong to any category; or the check box 39b indicating all images, the check boxes 38a and 39b being displayed in the classification check mark tool box 39 (step S32).

Subsequently, the check state of the classification check mark tool box 39 is determined (step S33). If the check box 39b denoting “ALL” is checked, all of image data items are read without distinction of category (step S34).

On the other hand, if any one or more segments in the check box 39a are checked in step S33 mentioned above, the classification file and the like in which classification information regarding the corresponding image is stored is read (step S35). Whether the classification information matches the category designated in the check box 39a, where the category of the corresponding image is selected, or whether the classification information matches the fact that the corresponding image does not belong to any category is determined (step S36). If NO, the process is returned to step S35 to read data of a classification file corresponding to the next image.

In step S36, if the classification information matches the category of the corresponding image or the fact that the corresponding image does not belong to any category, the image data is read (step S37).

The image data read in step S37 or the image data items read in step S34 are displayed as thumbnail images in the browse area 36 in the browse mode or in the thumbnail area 41 in the view mode or VLB mode (step S38). Consequently, only images of the designated category are narrowed to and are displayed (if the user designates images which do not belong to any category, the images are displayed).

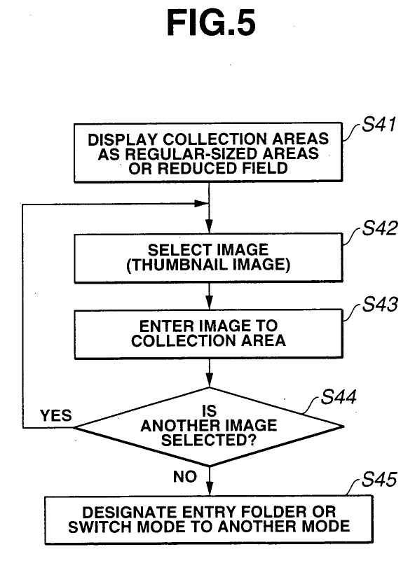

FIG. 5 is a flowchart showing the operation in the browse mode, the operation being performed mainly through the comparative selection means 20.

When the execution of the image processing program is started in the PC 2, or when a shift to the browse mode from another mode is operated, the collection areas are displayed as regular-sized areas (refer to the collection areas 38A and 38B in FIG. 9) or a reduced field (refer to the collection field 38 in FIG. 8) (step S41).

When necessary, the user selects any one of the thumbnail images displayed in the browse area 36 using the mouse 12 or the like (step S42).

The selected image is entered into the collection area A or B which the user designates (when he or she designates both the collection areas A and B, the selected image is entered into both the areas) (step S43). The entry is temporal. Only after the user clicks on the save button 38c, the image file is copied to the folder 15a. Thus, in step S43, the image is only temporarily entered as an image file to be copied.

Subsequently, whether another image is to be selected is determined (step S44). If YES, the process is returned to step S42, thus repeating the above-mentioned steps.

When another image is not selected, an entry folder is designated and the like so long as the user clicks on the save button 38c. If the user changes the mode to another mode, the mode shifting operation corresponding to the change is performed (step S45).

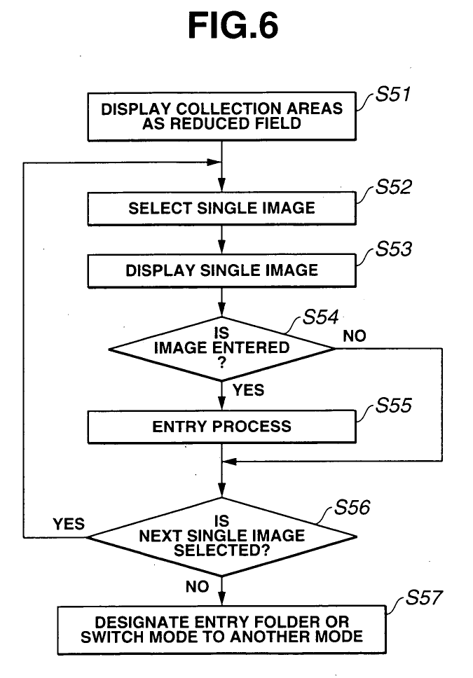

FIG. 6 is a flowchart showing the operation in the view mode or the full-screen display mode, the operation being performed mainly through the comparative selection means 20.

When the user operates to change the mode to the view mode or the full-screen display mode, the collection areas are reduced and displayed (refer to the collection field 38 in FIG. 10) (step S51).

Subsequently, the user selects one single image in the thumbnail area 41 via dragging and dropping (S52). The single image selected is displayed in the view area 42 (or in the full screen) (step S53).

Whether the displayed image is entered is determined (step S54). If YES, the image is entered into the collection area A or B which the user designates (step S55).

If the entry in step S55 is terminated, alternatively, if it is determined in step S54 that the entry is not performed, whether the next single image is selected is determined on the basis of whether another image in the thumbnail area 41 is selected, or whether the next-or-preceding image button 50 is operated (step S56), or the like. If YES, the process is returned to step S52, thus repeating the above-mentioned steps.

If the next single image is not selected, an entry folder is designated, or the mode is changed to another mode (step S57).

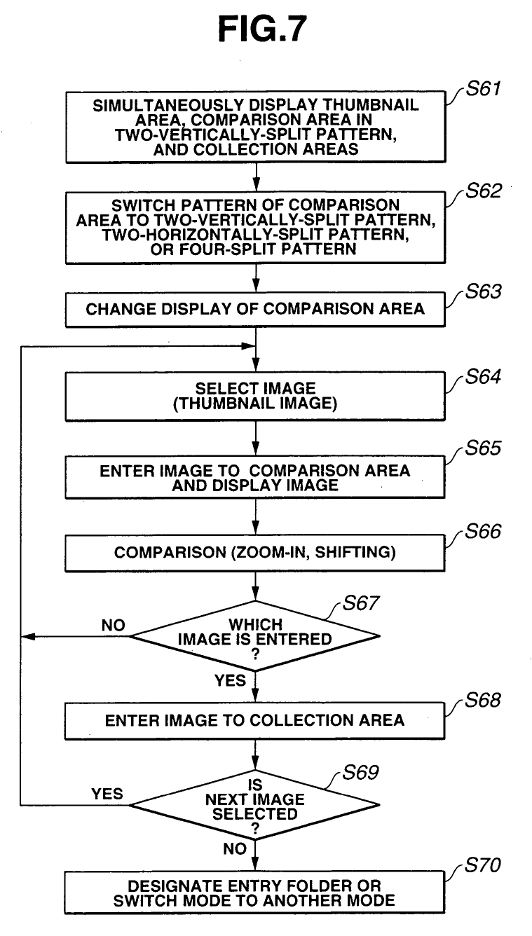

FIG. 7 is a flowchart showing the operation in the VLB mode, the operation being performed mainly through the comparative selection means 20.

When the user operates to change the mode to the VLB mode, the thumbnail area 41, the comparison area 51 in the two-image-vertically-split pattern, and the collection areas 38A and 38B are displayed simultaneously as shown in FIG. 11 (step S61). In this case, for example, the two-image-vertically-split pattern is set as an initial state obtained when the mode is changed to the VLB mode.

After that, when necessary, the user switches the pattern of the comparison area using the display-mode change button 57 (step S62). According to the first embodiment, the two-image-vertically-split pattern (refer to FIG. 11), the two-image-horizontally-split pattern, and the four-image-split pattern (refer to FIG. 12), namely, three kinds of patterns are preset as switchable comparison-area patterns. The pattern is not limited to the above examples.

If the pattern of the comparison area is switched to another pattern in step S62, the display is changed in accordance with the changing operation (step S63). If the pattern of the comparison area is not switched in step S62, the process proceeds to step S64 without particularly processing in steps S62 and S63.

Subsequently, when necessary, the user selects any of the thumbnail images displayed in the thumbnail area 41 (step S64).

The selected image is entered into the comparison area 51 and is displayed therein (step S65).

After a plurality of images are displayed in the comparison area 51, the user compares the images. In this instance, when necessary, the user zooms out or zooms in the images using the zoom-out button 46 or the zoom-in button 47, and/or moves the images using the moving-pointer set button 48. The images are processed in accordance with the operation (step S66).

Whether any of the images displayed in the comparison area 51 is entered into the collection area is determined (step S67). If NO, the process is returned to step S64, thus repeating the above-mentioned steps.

If the entry is performed in step S67, the image is entered into the collection area 38A or 38B which the user designates (step S68).

After that, whether the next image is selected is determined (step S69). If YES, the process is returned to step S64, thus repeating the above-mentioned steps.

On the other hand, if the next image is not selected, an entry folder is designated or the mode is changed to another mode (step S70).

When the above-mentioned image processing program is recorded in a computer-readable recording medium and another computer reads and runs the program, the program can permit the computer to similarly function.

Further, in the above description, the image processing program is run on the general purpose PC connected to the digital camera, thus realizing the image processing apparatus. The invention is not limited to the above arrangement. A dedicated image processing apparatus may be constructed such that the same functions as those of a computer, on which the image processing program runs, are realized by hardware.

According to the first embodiment, when several images are selected from among many images, the images are classified into categories. The images can be selected from image groups of the classified categories. Thus, desired images can be selected with simple operations.

Further, the process of classifying the images into the categories is performed on the PC which functions as an image processing apparatus and on which the image processing program runs. Accordingly, images, which are not previously classified through the digital camera, can also be processed. Advantageously, the versatility is high.

As the classification check box used to put an image into any of the categories, for example, the user can use colored segments or the like constituting the check box. Accordingly, the user can determine what categories are represented by what colors. Thus, the classification check box can be used for various purposes.

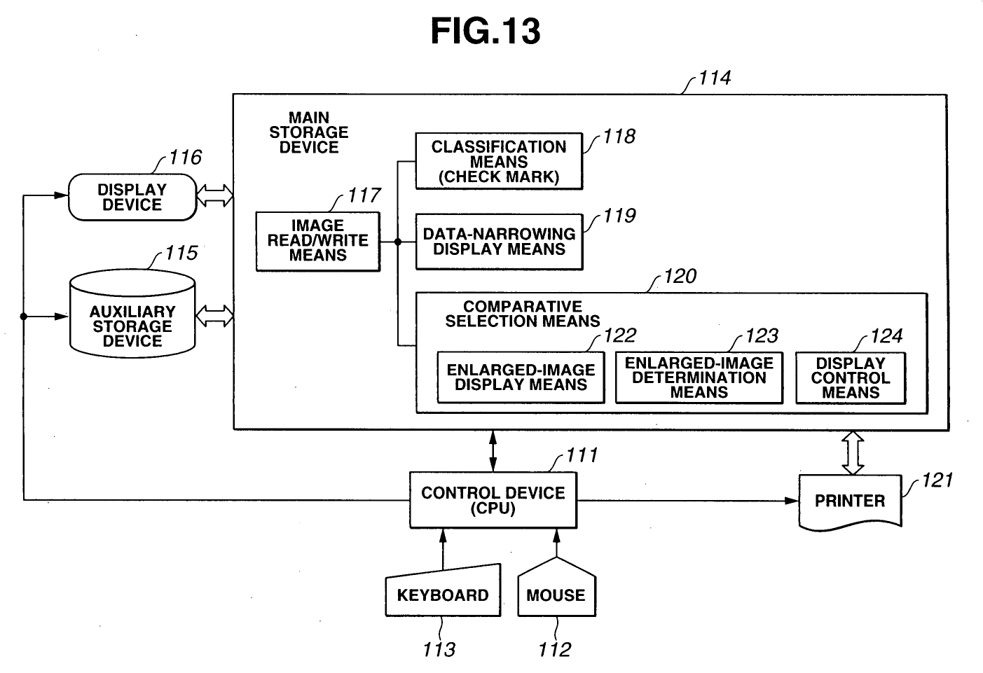

FIGS. 13 to 24E show a second embodiment of the present invention. FIG. 13 is a block diagram schematically showing the structure of a PC.

The PC comprises: a control device 111 comprising a CPU and the like and functioning as display control means, target image-space shifting means, and collective display set means; a mouse 112 serving as selection means for pointing a position on a screen of a display device 116, which will be described later, in order to perform the operation, the mouse 112 also serving as selective designation means; a keyboard 113 serving as selection means for character input, the keyboard 113 also serving as selective designation means; a main storage device 114 comprising a RAM and the like and serving as a work area of the control device 111; an auxiliary storage device 115 comprising a readable recording medium such as a hard disk in which a hierarchical file system having folders is constructed, and an image processing program and image data are recorded; and the display device 116 serving as display means for displaying window images and other various information items according to the image processing program in a visual form.

The PC connects to, for example, a printer 121. Under the control of the control device 111, image data transferred from the main storage device 114 can be printed through the printer 121.

In the main storage device 114, an operating system for controlling the fundamental operation of the PC and the image processing program running on the operating system are loaded such that the system and the program can be executed. In addition, various data items such as image data are loaded such that each data is processed.

The image processing program comprises: image read/write means 117 for reading image data from the auxiliary storage device 115, writing processed image data into the auxiliary storage device 115, and/or outputting the processed image data to the printer 121; classification means 118 for putting each image, stored in a predetermined folder in the auxiliary storage device 115, into any one of a plurality of preset categories; data-narrowing display means 119 for displaying only images belonging to at least one category in a predetermined display area in the display device 116 when one or more categories are designated among the categories of images classified through the classification means 118; and comparative selection means 120 for displaying two or more images read from the auxiliary storage device 115 through the image read/write means 117 in a predetermined display area in the display device 116 to make a comparison and selection therebetween.

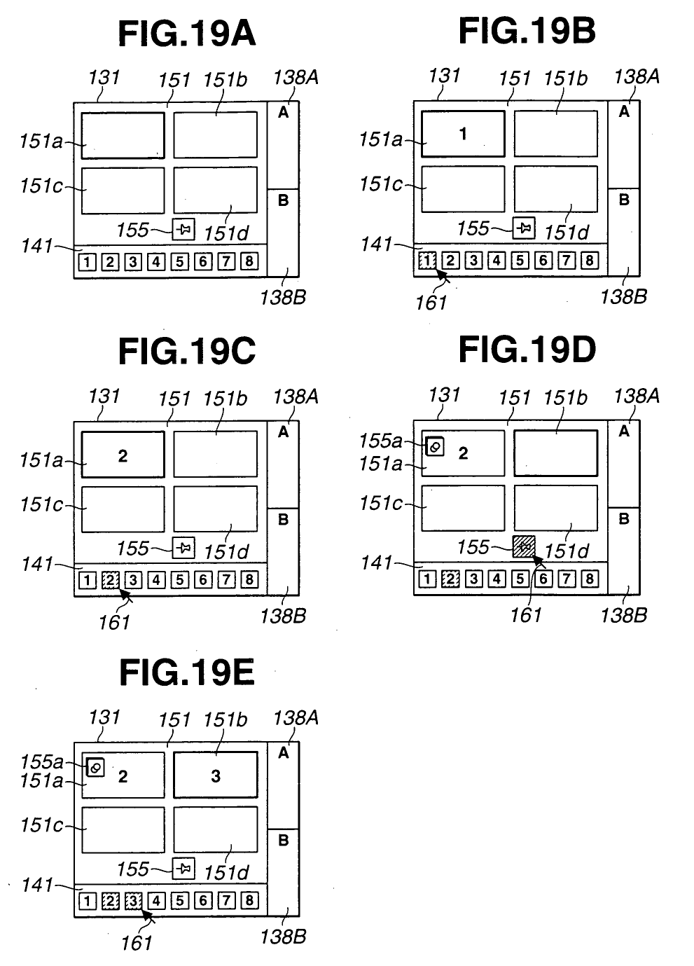

The comparative selection means 120 includes: enlarged-image display means 122 for enlarging a read image to display the enlarged image in a comparison area 151, which will be described later (refer to FIGS. 19A to 24E); enlarged-image determination means 123 for determining the image, displayed in the comparison area 151 through the enlarged-image display means 122, as a selection target in a collection area 138A or 138B, which will be described later (refer to FIGS. 19A to 24E); and display control means 124 serving as target image-space shifting means for controlling the operation for adding an image from a thumbnail area 141, which will be described later (refer to FIGS. 19A to 24E), to the comparison area 151 and also serving as collective-display set means. The add operation will be described in detail later.

In the PC on which the image processing program runs, a browse mode, a view mode, and a VLB (virtual light box) mode can be bidirectionally shifted to each other. The browse mode mainly displays an array of reduced images (thumbnail images). The view mode mainly enlarges and displays one single thumbnail image selected from thumbnail images in the thumbnail area 141, which is simultaneously displayed. The VLB mode mainly compares and selects between and among a relatively small number of images, for example, two or four images. Further, any of the above modes can be shifted to a full-screen display mode in which one single image is displayed in the whole of a screen of the display device 116. The full-screen display can also be returned to the preceding mode.

As for the VLB mode, specifically, which image is selected from a plurality of images obtained by shooting substantially the same scene with varied exposure, alternatively, which shot is selected from among similar shots, the VLB mode is often used.

Further, in the above respective modes and the full-screen display, displayed images can be classified into categories such that each image belongs to any one of the categories. When the displayed images (all images of all the categories may be displayed, or only images belonging to one or more categories designated may be displayed) are desired images, the images can be selectively distributed to the collection areas 138A and 138B.

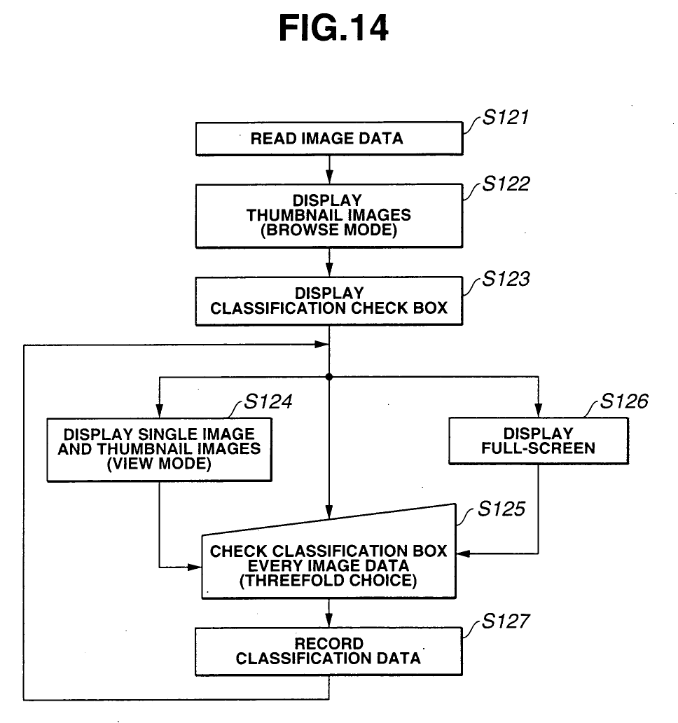

FIG. 14 is a flowchart showing the operation which is performed mainly through the classification means 118.

At the start of a process, image data is read from the auxiliary storage device 115 through the image read/write means 117 (step S121). In the browse mode, a folder and thumbnail images are displayed in a browse area (step S122). If the thumbnail images are recorded at the respective headers or the like of the corresponding image files, the thumbnail images are read and displayed. If a certain image file does not include any thumbnail image, a thumbnail image is formed from the corresponding image data and is then displayed.

Subsequently, a classification check box is displayed in each of the thumbnail images (step S123). The user selects any of segments constituting the classification check box to designate a category to which the corresponding image belongs.

When the browse mode is shifted to the view mode, the thumbnail images are displayed in the thumbnail area 141 and an image selected from among the displayed thumbnail images is displayed in a view area (if an image is not designated, the first image displayed in the thumbnail area 141 is displayed in the view area) (step S124). In the view area, the classification check box is also displayed.

When any of the thumbnail images is designated such that the corresponding image is displayed in the full screen, the designated image is displayed in the full screen (step S126). In the full screen display, the classification check box is also displayed.

Any of segments constituting the classification check box is checked in a threefold-choice manner every image data in any of the browse mode in step S123, the view mode in step S124, and the full screen display in step S126 (step S125). Classification data formed in the above step is stored as, for example, an exclusive classification file independent of the image file in the auxiliary storage device 115 (step S127). The operation for storing classification data is performed every image at each time the classification check box 30 is checked.

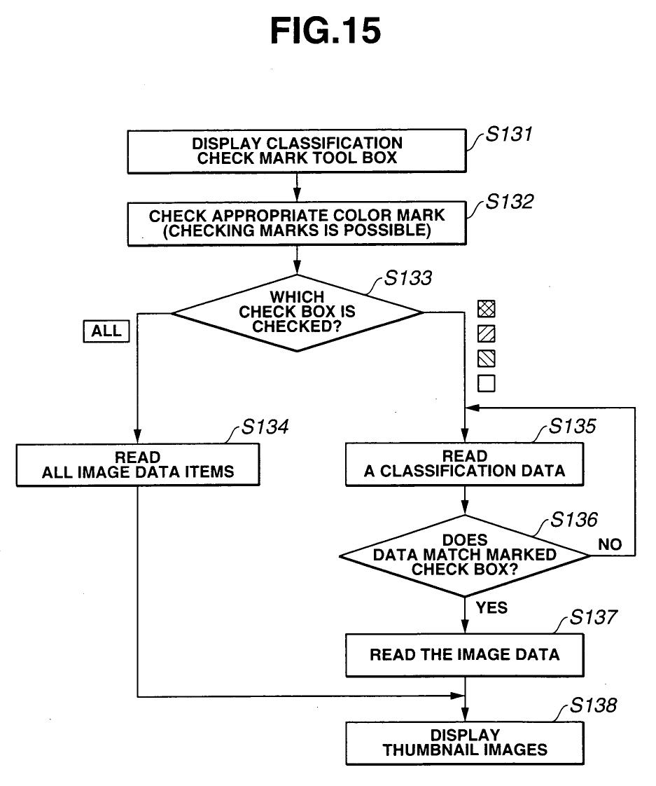

FIG. 15 is a flowchart showing the operation which is mainly performed through the data-narrowing display means 119.

At the start of a process, a classification check mark tool box is displayed (step S131). The classification check mark tool box is used to designate at least one category of images displayed in the browse area. Different from the classification check box described in FIG. 14, a plurality of check box segments can be simultaneously checked. The classification check mark tool box includes: a plurality of first check boxes indicating respective categories; a second check box indicating that the corresponding image does not belong to any category; and a third check box indicating that all images (ALL) are displayed regardless of the categories.

When necessary, the user checks any one or more check boxes among the first and second check boxes, or the third check box indicating all images in the classification check mark tool box (step S132).

Subsequently, the check state of the classification check mark tool box is determined (step S133). If the third check box denoting “ALL” is checked, all of image data items are read regardless of the categories (step S134).

On the other hand, if any one or more check boxes among the first and second check boxes are checked in step S133 mentioned above, a classification file, in which classification information regarding the corresponding image is stored, is read (step S 135). Vvhether the classification information matches the category designated in the selected check box, or whether the classification information matches the fact that the corresponding image does not belong to any category is determined (step S136). If NO, the process is returned to step S135 to read data of a classification file corresponding to the next image.

In step S136, if the classification information matches the category of the corresponding image or the fact that the corresponding image does not belong to any category, the image data is read (step S137).

The image data items read in step S137 or the image data items read in step S134 are displayed as thumbnail images in the browse area in the browse mode or in the thumbnail area 141 in the view mode or VLB mode (step S138). Consequently, only images of at least one designated category are narrowed to and displayed (if the user designates images which do not belong to any category, the images are narrowed to and displayed).

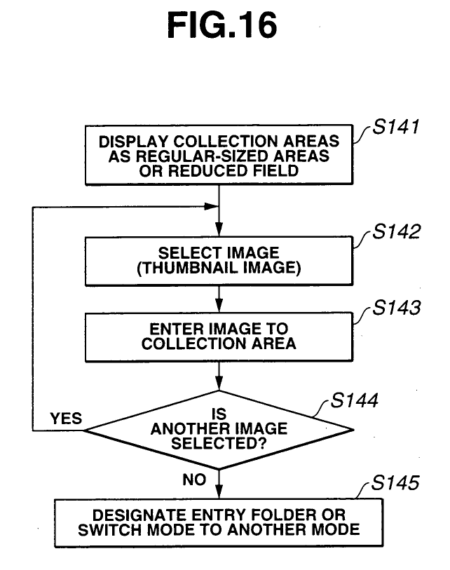

FIG. 16 is a flowchart showing the operation in the browse mode, the operation being performed mainly through the comparative selection means 120.

When the execution of the image processing program is started in the PC, or when a shift to the browse mode from another mode is operated, the collection areas 138A and 138B are displayed as regular-sized areas or a reduced field (step S141).

When necessary, the user selects any one of the thumbnail images displayed in the browse area using the mouse 112 (step S142).

The selected image is entered into the collection area 138A or 138B which the user designates (step S143). The entry is temporarily performed. Only after the user clicks on a save button, the image file is copied to a folder in which a selected image is stored. In step S143, only the entry of the image as an image file to be copied is performed.

Subsequently, whether another image is selected is determined (step S144). If YES, the process is returned to step S142, thus repeating the above-mentioned steps.

When another image is not selected, an entry folder is designated or the like so long as the user clicks on the save button. Alternatively, when the user changes the mode to another mode, the mode shift operation corresponding to the changing operation is performed (step S145).

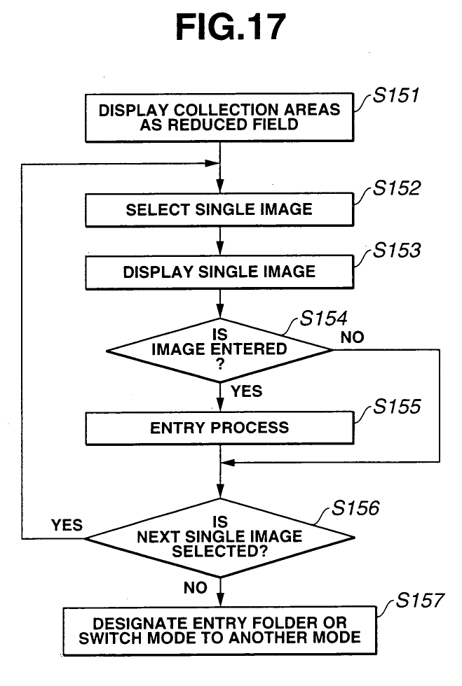

FIG. 17 is a flowchart showing the operation in the view mode or the full-screen display, the operation being performed mainly through the comparative selection means 120.

When the user operates to shift the mode to the view mode or the full-screen display, the collection areas 138A and 138B are displayed as a reduced field (step S151).

Subsequently, the user selects one single image in the thumbnail area 141 via dragging and dropping (S152) or the like. The single image selected is displayed in the view area (or in the full screen) (step S153).

Whether the displayed image is entered is determined (step S154). If YES, the image is entered into the collection area 138A or 138B which the user designates (step S155).

If the entry in step S155 is terminated, alternatively, if it is determined in step S154 that the entry is not performed, whether the next single image is selected is determined (step S156). If YES, the process is returned to step S152, thus repeating the above-mentioned steps.

If the next single image is not selected, an entry folder is designated, or the mode is changed to another mode (step S157).

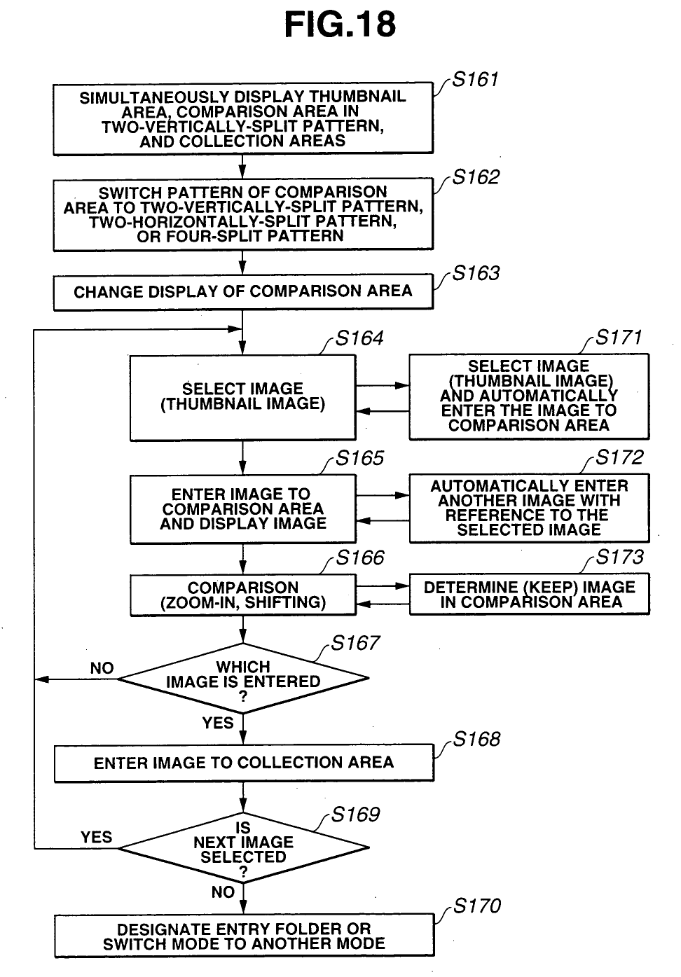

FIG. 18 is a flowchart showing the operation in the VLB mode, the operation being performed mainly through the comparative selection means 120.

When the user operates to change the mode to the VLB mode, the thumbnail area 141 as a first display area, the comparison area 151 in a two-image-vertically-split pattern as a second display area, and the collection areas 138A and 138B as third display areas are simultaneously displayed as shown in FIGS. 19A to 24E (step S161). In this case, for example, the two-image-vertically-split pattern is set as an initial state obtained when the mode is changed to the VLB mode.

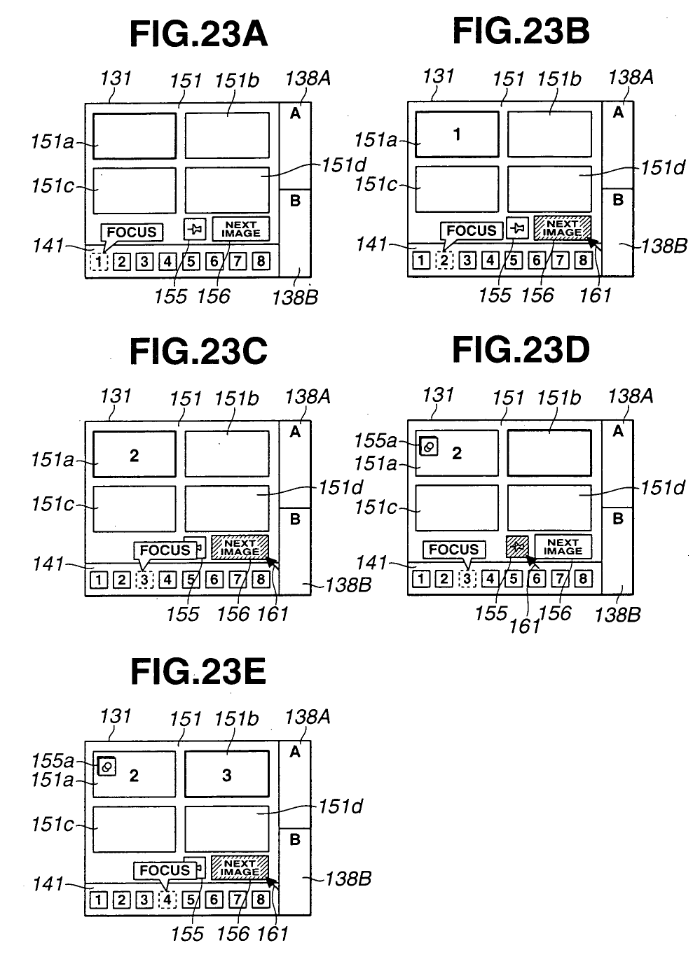

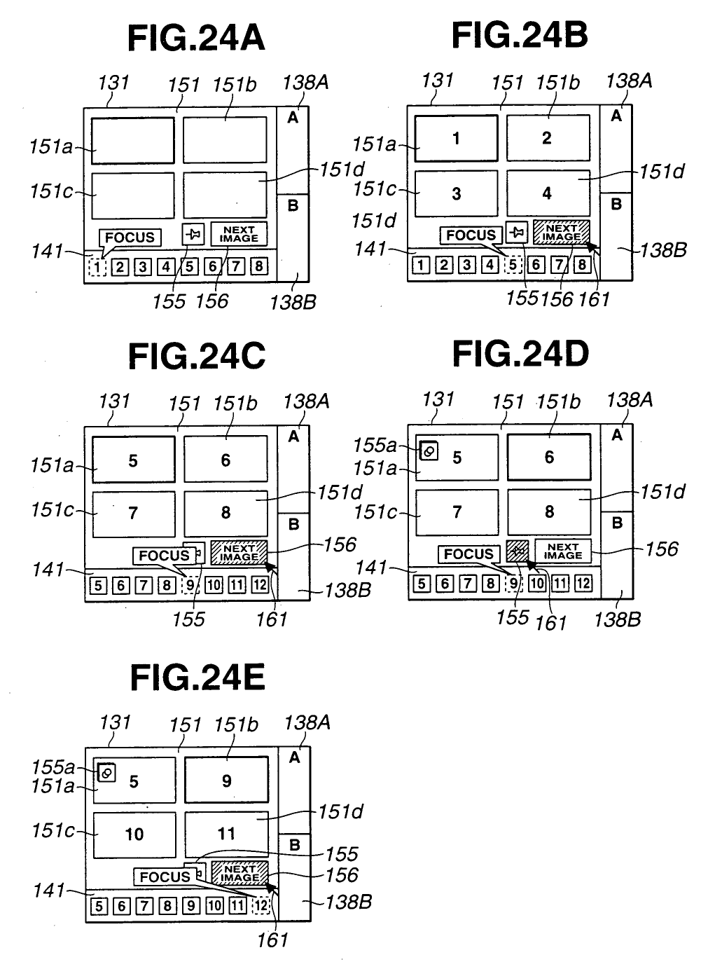

After that, when necessary, the user switches the pattern of the comparison area 151 serving as the second display area with a display-mode change button (S162). According to the second embodiment, the two-image-vertically-split pattern (refer to FIGS. 23A to 23E), a two-image-horizontally-split pattern, and a four-image-split pattern (refer to FIGS. 24A to 24E), namely, three kinds of patterns are preset as switchable patterns of the comparison area 151. The pattern is not limited to the above examples.

If the pattern of the comparison area 151 is switched to another pattern in step S162, the display is changed in accordance with the operation through the display control means 124 (step S163). If the pattern of the comparison area 151 is not switched in step S162, the process proceeds to step S164 without particularly processing in steps S162 and S163.

Subsequently, when necessary, the user selects any one of the thumbnail images displayed in the thumbnail area 141 using the mouse 112 via dragging and dropping or an image add button 156 (refer to FIGS. 23A to 23E and 24A to 24E) (step S164).

In this instance, so long as a selective-add-to-list check box, which will be described later, is checked, in addition to the selection by the drag and drop operation or the operation using the image add button 156 in step S164, the user single-clicks on any one of the thumbnail images displayed in the thumbnail area 141, so. that the corresponding thumbnail image can be automatically selected such that the image is added to the comparison area 151 (step S171).

The image selected in step S164 or step S171 is entered into the comparison area 151 and is displayed in the comparison area 151 through the enlarged-image display means 122 (step S165).

In this instance, so long as a collective-add-to-list check box, which will be described later, is checked, under the control of the display control means 124, the other images are automatically entered in the predetermined order with reference to the selected image (step S172).

In the automatic entry, if a pushpin mark 155a serving as display holding means is displayed on an image which has already been displayed in the comparison area 151 using a pushpin button 155, which will be described later (refer to FIGS. 19A to 19E), the corresponding image is kept without being changed to another one.

After a plurality of images are displayed in the comparison area 151 in step S165, or steps S165 and S172 (or step S173) mentioned above, the user compares the images. In this instance, when necessary, the user zooms out or zooms in the images using a zoom-out button or a zoom-in button, and/or moves the images using a moving pointer, which is set through a moving-pointer set button. The displayed images are processed in accordance with the operation (step S166).

During the comparison between the images performed by the user, a certain image is kept using the above-mentioned pushpin button 155 when necessary (step S173).

Whether any of the images displayed in the comparison area 151 is entered into the collection area 138A or 138B is determined (step S167). If NO, the process is returned to step S164, thus repeating the above-mentioned steps.

When the entry is performed in step S167, the image is entered through the enlarged-image determination means 123 into the collection area 138A or 138B, designated by the user (step S168).

After that, whether the next image is selected is determined (step S169). If YES, the process is returned to step S164, thus repeating the above-mentioned steps.

On the other hand, if the next image is not selected, an entry folder is designated or the mode is changed to another mode (step S170).

FIGS. 19A to 19E are diagrams showing the operation for adding an image from the thumbnail area 141 to the comparison area 151 in the VLB mode when the collective-add-to-list check box is OFF and the selective-add-to-list check box is ON.

In the VLB mode, as shown in the diagrams, the comparison area 151, the thumbnail area 141, and the collection areas 138A and 138B are displayed in a window 131.

The comparison area 151 includes four image display spaces 151a, 151b, 151c, and 151d in an array of 2×2 such that the spaces 151a, 151b, 151c, and 151d are arranged at the upper-left, upper-right, lower-left, and lower-right positions in that order. The arrangement of the image display spaces 151a to 151d has a cyclic order (a second order) such that 151a→151b→151c→151d→151a. Images selected in the thumbnail area 141 are sequentially displayed in the image display spaces 151a to 151d. The pushpin button 155 is displayed below the comparison area 151. The pushpin button 155 serves as display holding means for temporarily fastening an active image among the images displayed in the comparison area 151 so that the active image is not changed to another one.

In the thumbnail area 141, a plurality of thumbnail images stored in a predetermined folder are displayed in accordance with a predetermined sorting order (a first order). For example, eight thumbnail images are displayed.

In the VLB mode, the selective-add-to-list check box and the collective-add-to-list check box, which are not shown in detail in the diagrams, are further displayed such that the check boxes can be operated. As will be described later, when an image is added from the thumbnail area 141 to the comparison area 151, a thumbnail image displayed in the thumbnail area 141 is usually added to the comparison area 151 via dragging and dropping. In contrast to the usual add, when the selective-add-to-list check box is checked, each time the user only single-clicks the mouse 112 on an arbitrary thumbnail image displayed in the thumbnail area 141 to select the thumbnail image, he or she can add the image to the comparison area 151. On the other hand, when the collective-add-to-list check box is checked, the user merely drags and drops (or selects a thumbnail image with a single click, or operates the image add button 156, which will be described later, so long as the selective-add-to-list check box is checked) one of the thumbnail images displayed in the thumbnail area 141, so that the plurality of images in the thumbnail area 141 can be added to the comparison area 151 in a lump in accordance with the arrangement order in the thumbnail area 141.

It is assumed that when the mode is shifted to the VLB mode, a state shown in FIG. 19A is set as an initial state. In other words, any image is not displayed in the comparison area 151 and first to eighth images are displayed in the thumbnail area 141 in accordance with the predetermined sorting order. In the collection areas 138A and 138B, images selected in another mode may be entered. Alternatively, no entry is also available. In this initial state, among the plurality of image display spaces 151a to 151d, the first image display space 151a is set to be active as a target image display space. In the target image display space, an image is enclosed with, for example, a red frame, so that the image is visually shown as an active image.

It is assumed that in the above-mentioned initial state, the user operates the mouse 112, for example, points at the first image of the images displayed in the thumbnail area 141 using a pointer 161 serving as selection means and also serving as selective designation means and single-clicks thereon as shown in FIG. 19B.

As shown in FIG. 19B, the first image is displayed in the first image display space 151a which is active. At this time, any image is not displayed in each of the other image display spaces 151b to 151d. Further, the first thumbnail image, displayed in the thumbnail area 141, corresponding to the first image displayed in the comparison area 151 is highlighted. Thus, it is known that the highlighted image is currently displayed in the comparison area 151.

After that, it is assumed that as shown in FIG. 19C, the user moves the position of the pointer 161 to point the second image displayed in the thumbnail area 141 and then single-clicks on the second image.

As shown in FIG. 19C, the second image is written over the image in the first image display space 151a which is still active, so that the second image is displayed in this space 151a. At this time, any image is not;displayed in each of the other image display spaces 151b to 151d. In the thumbnail area 141, the second thumbnail image is, for example, highlighted in place of the first thumbnail image.

It is assumed that as shown in FIG. 19D, the user moves the position of the pointer 161 to point at the pushpin button 155 and then single-clicks thereon.

Then, the pushpin mark 155a is displayed on the second image in the first image display space 151a which is active, thus holding the second image such that another image is not displayed in the first image display space 151a. An active target image display space is automatically shifted to the second image display space 151b. The display of the second image is held in the first image display space 151a until the pushpin mark 155a is released.

It is assumed that as shown in FIG. 19E, the user moves the position of the pointer 161 to point at the third image displayed in the thumbnail area 141 and then single-clicks thereon.

The third image is displayed in the second image display space 151b which is active. At this time, the second image is still displayed in the first image display space 151a and any image is not displayed in each of the other image display spaces 151c and 151d. Further, the second thumbnail image in the thumbnail area 141 is still, for example, being highlighted and the third thumbnail image is also highlighted.

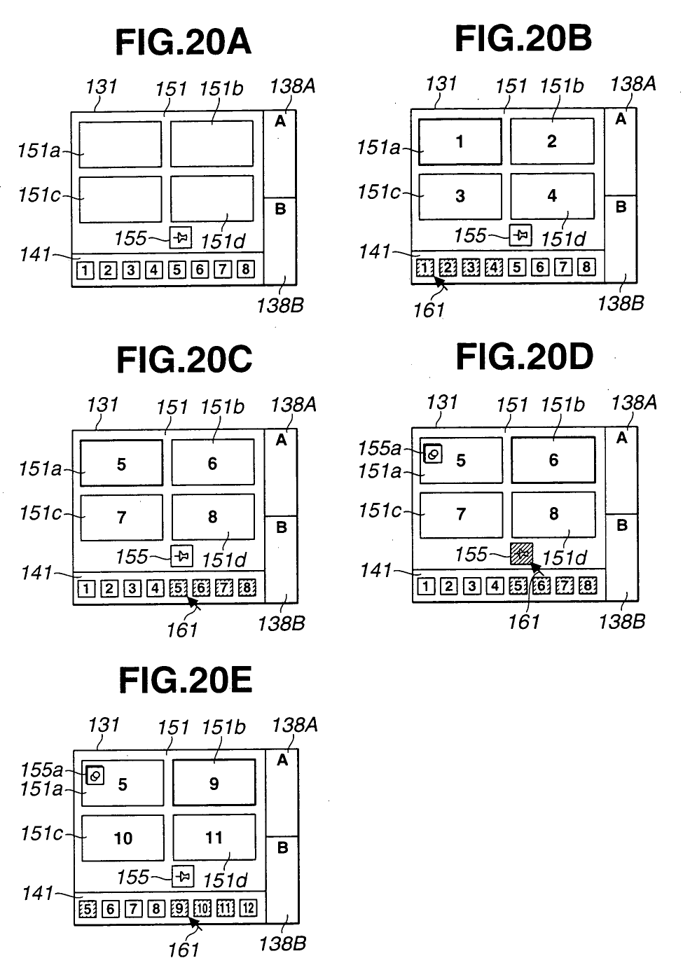

FIGS. 20A to 20E are diagrams showing the operation for adding an image from the thumbnail area 141 to the comparison area 151 in the VLB mode when the collective-add-to-list check box is ON and the selective-add-to-list check box is ON.

An initial state shown in FIG. 20A is the same as that shown in FIG. 19A mentioned above.

In this initial state, when the user points at the first image displayed in the thumbnail area 141 and then single-clicks thereon, the first image is displayed in the first image display space 151a which is active and the second, third, and fourth images are further displayed in the second, third, and fourth image display spaces 151b, 151c, and 151d, respectively, as shown in FIG. 20B.

As mentioned above, so long as the collective-add-to-list check box is ON, when the user only single-clicks on one image in the thumbnail area 141, the corresponding image and subsequent images are automatically displayed such that the image display spaces 151a to 151d in the comparison area 151 are filled with the respective images.

At this time, the first to fourth thumbnail images in the thumbnail area 141 are, for example, highlighted so that it is known that the highlighted images are currently displayed in the comparison area 151.

Subsequently, it is assumed that the user moves the position of the pointer 161 to point at the fifth image displayed in the thumbnail area 141, as shown in FIG. 20C, and then single-clicks on the fifth image.

Then, as shown in FIG. 20C, the fifth image is written over the image displayed in the first image display space 151a which is still active, so that the fifth image is displayed in the first image display space 151a. Moreover, the sixth, seventh, and eighth images are written over the images displayed in the second, third, and fourth image display spaces 151b, 151c, and 151d, respectively. Thus, the sixth, seventh, and eighth images are displayed in the second, third, and fourth image display spaces 151b, 151c, and 151d, respectively.

At this time, the fifth to eighth images in the thumbnail area 141 are, for example, highlighted so that it is known that the highlighted images are currently displayed in the comparison area 151.

Here, it is assumed that the user moves the pointer of the pointer 161 to point at the pushpin button 155 as shown in FIG. 20D and then clicks thereon.

Then, the pushpin mark 155a is displayed on the fifth image in the first image display space 151a which is active, such that the fifth image is held. An active target image display space is automatically shifted to the second image display space 151b.

Subsequently, it is assumed that the user moves the position of the pointer 161, scrolls a plurality of thumbnail images displayed in the thumbnail area 141, points at a ninth image in the thumbnail area 141 as shown in FIG. 20E, and then single-clicks on the ninth image.

Then, the ninth image is written over the image in the second image display space 151b which is active, so that the ninth image is displayed in the second image display space 151b. Moreover, tenth and eleventh images are written over the images in the third and fourth image display spaces 151c and 151d, respectively, so that the tenth and eleventh images are displayed in the third and fourth image display spaces 151c and 151d, respectively. At this time, the fifth image held in the first image display space 151a is continuously displayed without being changed to another one. Therefore, the fifth, ninth, tenth, and eleventh thumbnail images in the thumbnail area 141 are, for example, highlighted.

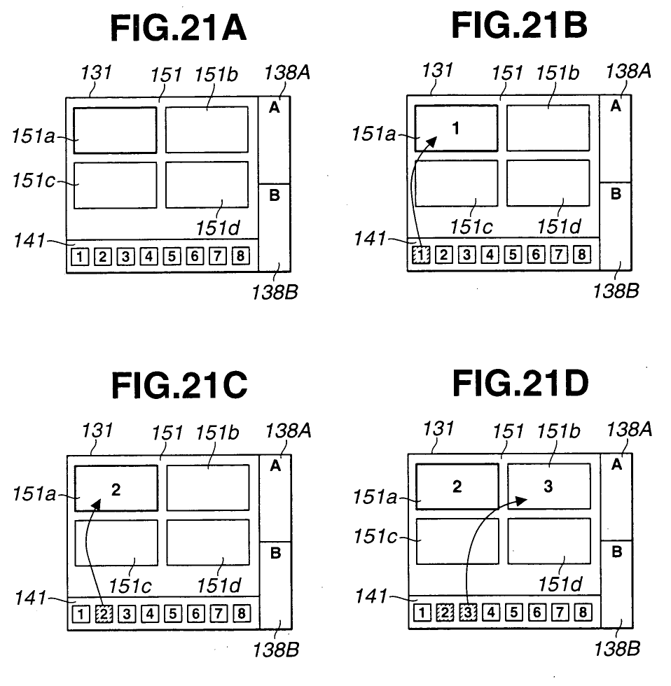

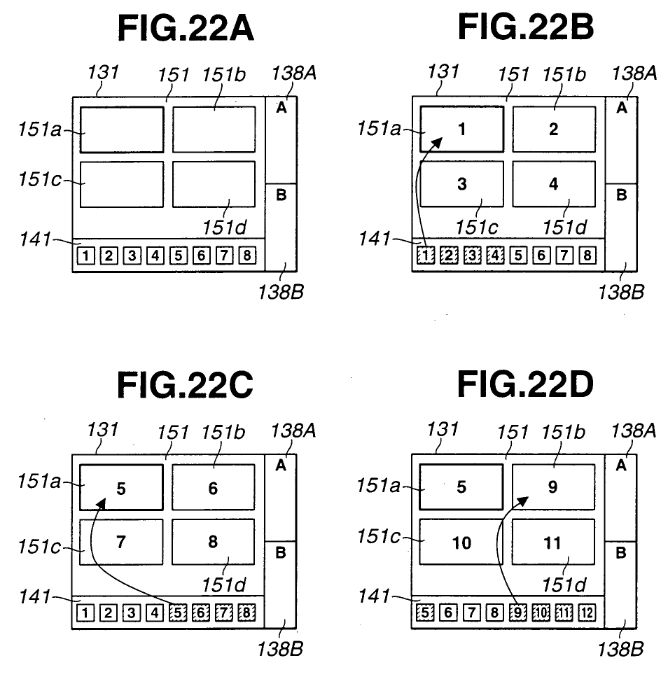

Subsequently, the operation during the OFF state of the selective-add-to-list check box will now be described with reference to FIGS. 21A to 22D. As described with reference to FIGS. 19A to 20E, during the ON state of the selective-add-to-list check box, the user single-clicks on any one of the thumbnail images displayed in the thumbnail area 141, so that the image is added to the comparison area 151. During the OFF state of the selective-add-to-list check box, the user adds a thumbnail image displayed in the thumbnail area 141 to the comparison area 151 via dragging and dropping.

Even in the OFF state of the selective-add-to-list check box, the user double-clicks on a thumbnail image displayed in the thumbnail area 141, resulting in the similar operation as that obtained by single-click on the thumbnail image in the ON state of the selective-add-to-list check box.

FIGS. 21A to 21D are diagrams showing the operation for adding an image from the thumbnail area 141 to the comparison area 151 in the VLB mode when the collective-add-to-list check box is OFF and the selective-add-to-list check box is OFF.

An initial state shown in FIG. 21A is the same as that shown in FIG. 19A.

In the initial state, the user drags the first image displayed in the thumbnail area 141 and drops it on the first image display space 151a in the comparison area 151. Then, as shown in FIG. 21B, the first image is displayed in the first image display space 151a which is active. In the case shown in FIG. 21A to 21D, since the collective-add-to-list check box is OFF, no image is displayed in each of the other image display spaces 151b to 151d.

Subsequently, the user drags the second image displayed in the thumbnail area 141 and drops it on the first image display space 151a in the comparison area 151. Then, as shown in FIG. 21C, the second image is written over the image in the first image display space 151a which is active, so that the second image is displayed in the space 151a.

When the user desires to hold the second image in the first image display space 151a and display another image, for example, the third image in the comparison area 151, the user drags the third image displayed in the thumbnail area 141 and then drops it on the second image display space 151b. Then, as shown in FIG. 21D, the third image is displayed in the second image display space 151b. At this time, the first image display space 151a is still active. The active state as a target display area is not shifted to the second image display space 151b. To shift the active state as a target display area to another space, the user needs to perform the shift operation separately.

FIGS. 22A to 22D are diagrams showing the operation for adding an image from the thumbnail area 141 to the comparison area 151 in the VLB mode when the collective-add-to-list check box is ON and the selective-add-to-list check box is OFF.

An initial state shown in FIG. 22A is the same as that shown in FIG. 21A mentioned above.

In the initial state, the user drags the first image displayed in the thumbnail area 141 and then drops it on the first image display space 151a in the comparison area 151. Then, as shown in FIG. 22B, the first image is displayed in the first image display space 151a which is active. Since the collective-add-to-list check box is ON, the second, third, and fourth images in the thumbnail area 141 are displayed in the second, third, and fourth image display spaces 151b, 151c, and 151d, respectively.

Subsequently, the user drags the fifth image displayed in the thumbnail area 141 and then drops it on the first image display space 151a in the comparison area 151. Then, as shown in FIG. 22C, the fifth image is written over the image in the first image display space 151a which is active, so that the fifth image is displayed in the space 151a. Moreover, the sixth, seventh, and eighth images in the thumbnail area 141 are written over the images in the second, third, and fourth image display spaces 151b, 151c, and 151d, respectively, so that the sixth to eighth images are displayed in the second to fourth spaces 151b to 151d, respectively.

When the user desires to, with the fifth image being held in the first image display space 151a, display other images, for example, the ninth and subsequent images in the thumbnail area 141 in the other spaces, the user drags the ninth image displayed in the thumbnail area 141 and then drops it on the second image display space 151b in the comparison area 151. Then, as shown in FIG. 22D, the ninth, tenth, and eleventh images are written over the images in the second, third, and fourth image display spaces 151b, 151c, and 151d, respectively. Thus, the ninth to eleventh images are displayed in the second to fourth image display spaces 151b to 151d, respectively.

As shown in FIGS. 23A to 23E and FIGS. 24A to 24E, the image add button 156 (not shown in FIGS. 19A to 22D) is displayed at, for example, the right of the pushpin button 155. FIGS. 23A to 24E show the operation for adding an image to the comparison area 151 using the image add button 156.

FIGS. 23A to 23E are diagrams showing the operation for adding an image from the thumbnail area 141 to the comparison area 151 using the image add button 156 in the VLB mode when the collective-add-to-list check box is OFF.

An initial state shown in FIG. 23A is substantially the same as that shown in FIG. 19A mentioned above. The first image in the thumbnail area 141 is currently focused (focusing indicating that the focused image is a target image to be subsequently added to the comparison area 151) and the currently focused image is shown by a frame or an arrow. For example, when the user points at the image add button 156 with the pointer 161, a focusing target is automatically displayed. The display is continued for a predetermined period.

In the above-mentioned state in which the first image is focused, the user single-clicks on the image add button 156. Then, as shown in FIG. 23B, the first image is displayed in the first image display space 151a which is active and the focus position is automatically shifted to the second image in the thumbnail area 141.

In this state, the user further single-clicks on the image add button 156. Then, as shown in FIG. 23C, the second image is written over the image in the first image display space 151a which is active, so that the second image is displayed in the space 151a. The focus position is automatically shifted to the third image in the thumbnail area 141.

In this instance, it is assumed that the user moves the position of the pointer 161 to point at the pushpin button 155 as shown in FIG. 23D and then single-clicks thereon.

Then, the pushpin mark 155a is displayed on the second image in the first image display space 151a which is active, so that the second image is held. The active state as a target image display space is automatically shifted to the second image display space 151b.

In this state, the user again moves the position of the pointer 161 to point at the image add button 156 and then single-clicks thereon. Then, as shown in FIG. 23E, the third image is displayed in the second image display space 151b which is active and the focus position is automatically shifted to the fourth image in the thumbnail area 141.

FIGS. 24A to 24E are diagrams showing the operation for adding an image from the thumbnail area 141 to the comparison area 151 using the image add button 156 in the VLB mode when the collective-add-to-list check box is ON.

An initial state shown in FIG. 24A is the same as that shown in FIG. 23A.

In this state in which the first image is focused, the user single-clicks on the image add button 156. Then, as shown in FIG. 24B, the first image is displayed in the first image display space 151a. The second, third, and fourth images are also displayed in the second, third, and fourth image display spaces 151b, 151c, and 151d, respectively. Further, the focus position is automatically shifted to the fifth image in the thumbnail area 141.

In this state, the user further single-clicks on the image add button 156. Then, as shown in FIG. 24C, the fifth image is written over the image in the first image display space 151a, so that the fifth image is displayed in the space 151a. The sixth, seventh, and eighth images are also written over the images in the second, third, and fourth image display spaces 151b, 151c, and 151d, respectively, so that the sixth, seventh, and eighth images are displayed in the spaces 151b, 151c, and 151d, respectively. Further, the focus position automatically moves to the ninth image in the thumbnail area 141.

In this instance, it is assumed that the user moves the position of the pointer 161 to point at the pushpin button 155 and then single-clicks thereon as shown in FIG. 24D.

The pushpin mark 155a is displayed on the fifth image in the first image display space 151a which is active, so that the fifth image is held. The active target image display space is automatically shifted to the second image display space 151b.

In this state, the user again moves the position of the pointer 161 to point at the image add button 156 and then single-clicks thereon. Then, as shown in FIG. 24E, the ninth image is written over the image in the second image display space 151b which is active, so that the ninth image is displayed in the space 151b. The tenth and eleventh images are written over the images in the third and fourth image display spaces 151c and 151d, respectively, so that the tenth and eleventh images are displayed in the spaces 151c and 151d, respectively. Further, the focus position automatically moves to the twelfth image in the thumbnail area 141.

The operation for selecting and determining an image using the mouse 112 has been mainly described. An image can also be similarly selected and determined using the keyboard 113 or another device.

When the above-described image processing program is recorded in a computer-readable recording medium and another computer reads and runs the program, the program can permit the computer to similarly function.

Further, in the above description, the image processing program is run on the general purpose PC connected to a digital camera, thus realizing the image processing apparatus. The invention is not limited to the above arrangement. A dedicated image processing apparatus can be constructed such that the same functions as those of a computer, on which the image processing program runs, are realized by hardware.

According to the second embodiment, several images among many images can be compared to each other with efficiency, so that a desired image can be selected.

In the ON state of the collective-add-to-list check box, when the user selects only one image, a plurality of images are added to the comparison area in a lump. Thus, the images can be added with efficiency.

In the selection of one image, the user can select any operation from among the single-click on a thumbnail image, the drag-and-drop of the thumbnail image to the comparison area, and the operation of the image add button. That is, the user can select the operation which is the easiest for the user. Thus, the flexibility of user interface is increased.

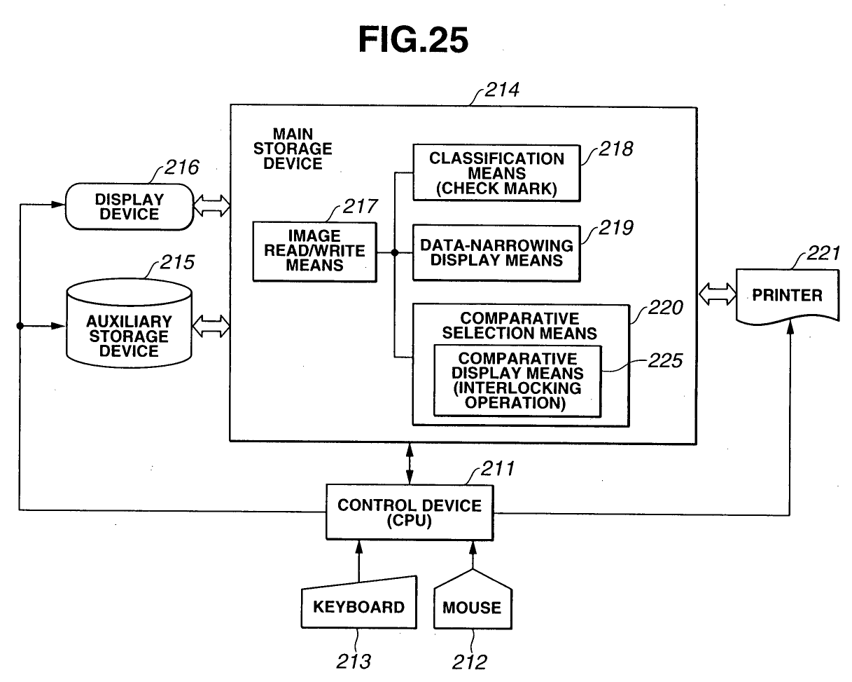

FIGS. 25 to 33D show a third embodiment of the present invention. FIG. 25 is a block diagram schematically showing the structure of a PC.

The PC comprises: a control unit 211 comprising a CPU; a mouse 212 for pointing at a position on a screen of a display device 216, which will be described later, in order to perform the operation; a keyboard 213 used for character input; a main storage device 214 comprising a RAM and serving as a work area of the control unit 211; an auxiliary storage device 215 comprising a readable recording medium such as a hard disk in which a hierarchical file system having folders is constructed, and an image processing program and image data are recorded; and the display device 216 for displaying window images and other various information items according to the image processing program in a visual form.

The PC connects to, for example, a printer 221. Under the control of the control unit 211, image data transferred from the main storage device 214 can be printed through the printer 221.

In the main storage device 214, an operating system for controlling the fundamental operation of the PC and the image processing program running on the operating system are loaded such that the system and the program can be executed. In addition, various data items such as image data are loaded such that each data is processed.

The image processing program comprises: image read/write means 217 for reading image data from the auxiliary storage device 215, writing processed image data into the auxiliary storage device 215, and/or outputting the processed image data to the printer 221; classification means 118 for putting an image, stored in a predetermined folder in the auxiliary storage device 215, into any one of a plurality of preset categories; data-narrowing display means 219 for displaying only images belonging to at least one category in a predetermined display area in the display device 216 when one or more categories are designated among the categories of images classified through the classification means 218; and comparative selection means 220 for displaying two or more images read from the auxiliary storage device 215 through the image read/write means 217 in a predetermined display area in the display device 216 to make a comparison and selection therebetween. The comparative selection means 220 also serves as display control means, image processing means, display scaling means, and display-image moving means.

The comparative selection means 220 includes comparative display means 225. The comparative display means 225 serves as comparison-image-size adjusting means for reading a plurality of images for comparison and displaying the images in the display device 216 and, when necessary, adjusting an image so as to uniform the display size of a main subject. The comparative display means 225 also serves as interlocking means for processing the images displayed as comparison images such that the images are operatively interlocked with each other when an arbitrary image is zoomed in or zoomed out, and/or is moved.

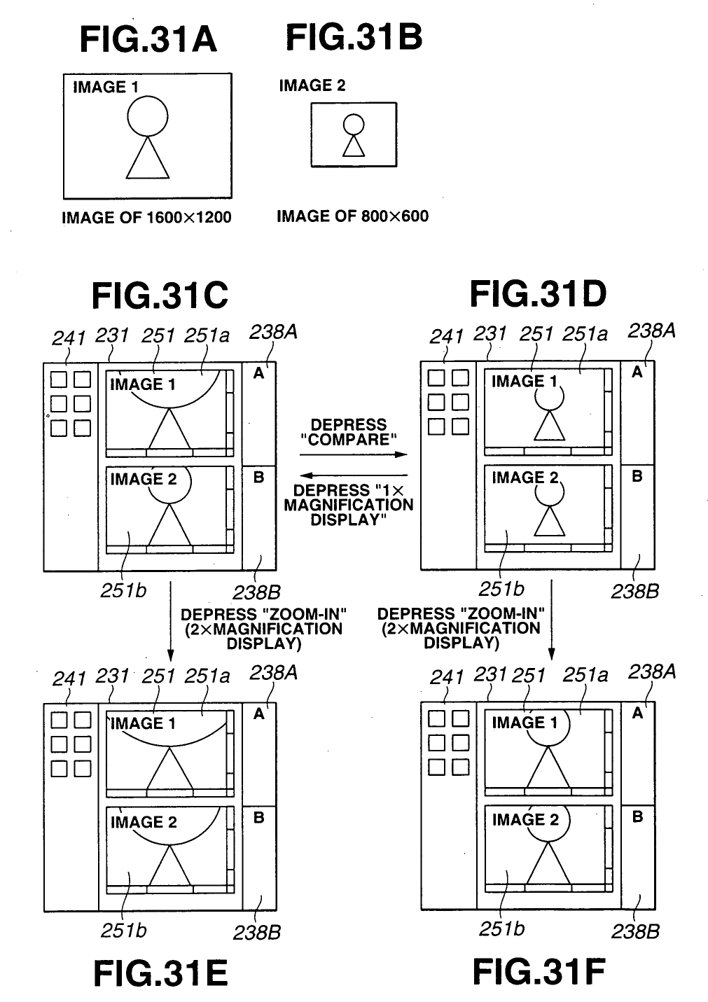

In the PC on which the image processing program runs, a browse mode, a view mode, and a VLB (virtual light box) mode can be bidirectionally shifted to each other. In the browse mode, an array of reduced images (thumbnail images) is mainly displayed. In the view mode, one single thumbnail image is selected from a thumbnail area 241 (refer to FIGS. 31A to 33D) displayed simultaneously with the browse mode, the selected image is enlarged, and the enlarged image is mainly displayed. In the VLB mode, relatively small number of images, for example, two or four images are mainly compared to each other to select one image. Further, any of the above modes can be shifted to a full-screen display mode in which one single image is displayed in the whole of a screen of the display device 216. The full-screen display can also be returned to the preceding mode.

As for the VLB mode, specifically, which image is selected from a plurality of images obtained by shooting substantially the same scene with varied exposure, alternatively, which shot is selected from among similar shots, the VLB mode is often used.

Further, in the above respective modes and the full-screen display, displayed images can be classified into categories such that each image belongs to any one of the categories. When the displayed images (all images of all the categories may be displayed, and when one or more categories are designated, only images belonging to the designated categories may be displayed) are desired images, the images can be selectively distributed to collection areas 238A and 238B, which will be described later (refer to FIGS. 31A to 33D).

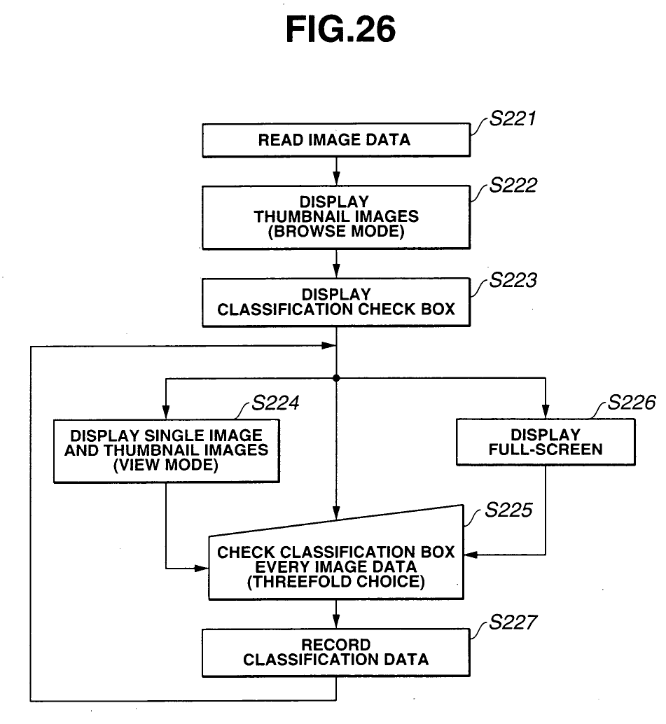

FIG. 26 is a flowchart showing the operation which is performed mainly through the classification means 218.

At the start of a process, image data is read from the auxiliary storage device 215 through the image read/write means 217 (step S221). In the browse mode, a folder and thumbnail images are displayed in a browse area (step S222). If the thumbnail images are recorded at the respective headers of the corresponding image files, the thumbnail images are read therefrom and are displayed. If a certain image file does not include any thumbnail image, a thumbnail image is formed from the corresponding image data and is then displayed.

Subsequently, a classification check box is displayed in each of the thumbnail images (step S223). The user alternatively selects any of segments constituting the classification check box to designate a category to which the corresponding image belongs.

When the browse mode shifts to the view mode, the thumbnail images are displayed in the thumbnail area 241 and an image selected from among the thumbnail images is displayed in a view area (if any image is not designated, the first image displayed in the thumbnail area 241 is displayed in the view area) (step S224). The classification check box is also displayed in the view area.

When any of the thumbnail images is designated such that the corresponding image is displayed in the full screen, the image is displayed in the full screen (step S226). In the full screen display, the classification check box is also displayed.

Any of segments constituting the classification check box is checked in a threefold-choice manner every image data in any of the browse mode in step S223, the view mode in step S224, and the full screen display in step S226 (step S225). Then, classification data formed in the above step is stored as, for example, a dedicated classification file independent of the image file in the auxiliary storage device 215 (step S227). The operation for storing classification data is performed every image. Each time the classification check box is checked, the storage operation is performed.

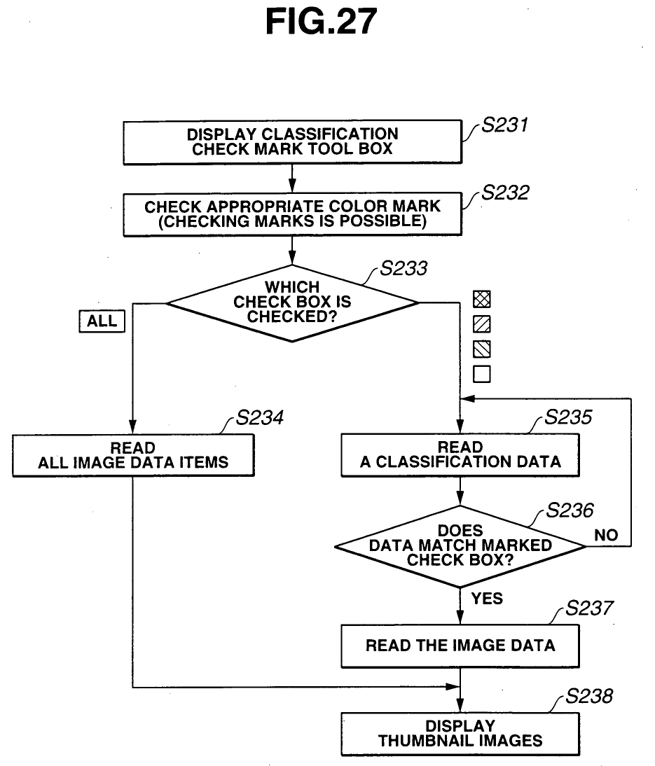

FIG. 27 is a flowchart showing the operation which is mainly performed through the data-narrowing display means 219.

At the start of a process, a classification check mark tool box is displayed (step S231). The classification check mark tool box is used to designate one category of images displayed in the browse area. Different from the classification check box described in FIG. 26, a plurality of check box segments can be simultaneously checked. The classification check mark tool box includes: a plurality of first check boxes indicating respective categories; a second check box indicating that the corresponding image does not belong to any category; and a third check box indicating that all images (ALL) are displayed regardless of the categories.

When necessary, the user checks any one or more check boxes among the first and second check boxes, or the third check box indicating all images, the first to third check boxes being displayed in the classification check mark tool box (step S232).

Subsequently, the check state of the classification check mark tool box is determined (step S233). If the third check box denoting “ALL” is checked, all of image data items are read regardless of the categories (step S234).

On the other hand, if any one or more check boxes in the first and second check boxes are checked in step S233 mentioned above, a classification file, in which classification information regarding the corresponding image is stored, is read (step S235). Whether the classification information matches the fact that the corresponding image belongs to the category designated in the selected check box, or whether the classification information matches the fact that the corresponding image does not belong to any category is determined (step S236). If NO, the process is returned to step S235 to read data of a classification file corresponding to the next image.

In step S236 mentioned above, if the classification information matches the category of the corresponding image or the fact that the corresponding image does not belong to any category, the image data is read (step S237).

The image data items read in step S237 or the image data items read in step S234 are displayed as thumbnail images in the browse area in the browse mode or in the thumbnail area 241 in the view mode or VLB mode (step S238). Consequently, only images of at least one designated category are narrowed to and displayed (if the user designates images which do not belong to any category, the images are narrowed to and displayed).

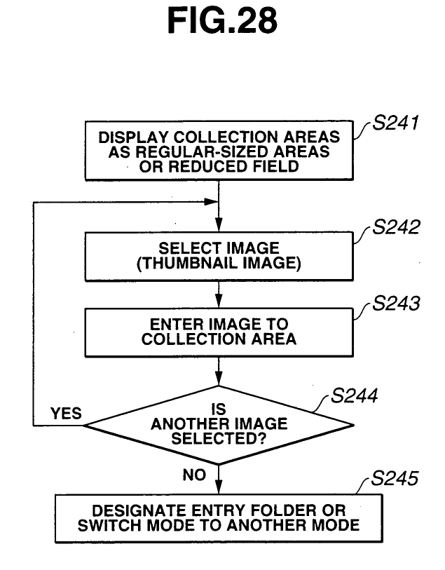

FIG. 28 is a flowchart showing the operation in the browse mode, the operation being performed mainly through the comparative selection means 220.

When the execution of the image processing program is started in the PC, or when a certain mode is shifted to the browse mode, the collection areas 238A and 238B are displayed as regular-sized areas or a reduced field (step S241).

When necessary, the user selects any one of the thumbnail images displayed in the browse area using the mouse 212 (step S242).

The selected image is entered into the collection area 238A or 238B which the user designates (step S243). The entry is temporarily performed. Only after the user clicks on a save button, the corresponding image file is copied to a folder in which a selected image is stored. Thus, in step S243, the image is only temporarily entered as an image file to be copied.

Subsequently, whether another image is selected is determined (step S244). If YES, the process is returned to step S242, thus repeating the above-mentioned steps.

When another image is not selected, an entry folder is designated and the like so long as the user clicks on the save button. Alternatively, when the user changes the mode to another mode, the mode shift operation corresponding to the operation is performed (step S245).

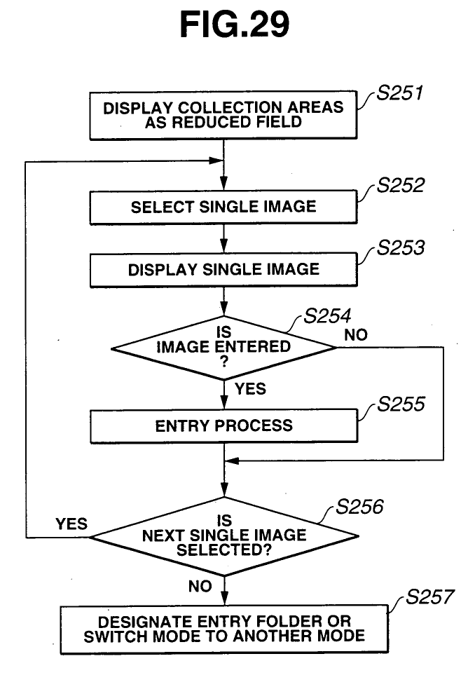

FIG. 29 is a flowchart showing the operation in the view mode or the full-screen display, the operation being performed mainly through the comparative selection means 220.

When the user changes the mode to the view mode or the full-screen display mode, the collection areas 238A and 238B are displayed as a reduced field (step S251).

Subsequently, the user selects one single image in the thumbnail area 241 via dragging and dropping (S252). Then, the single image selected is displayed in the view area (or in the full screen) (step S253).

Whether the displayed image is entered is determined (step S254). If YES, the image is entered into the collection area 238A or 238B according to the user's designation (step S255).

If the entry in step S255 is terminated, alternatively, if it is determined in step S254 that the image is not entered, whether the next single image is selected is determined (step S256). If YES, the process is returned to step S252, thus repeating the above-mentioned steps.

If the next single image is not selected, an entry folder is designated. Alternatively, the mode is changed to another mode (step S257).

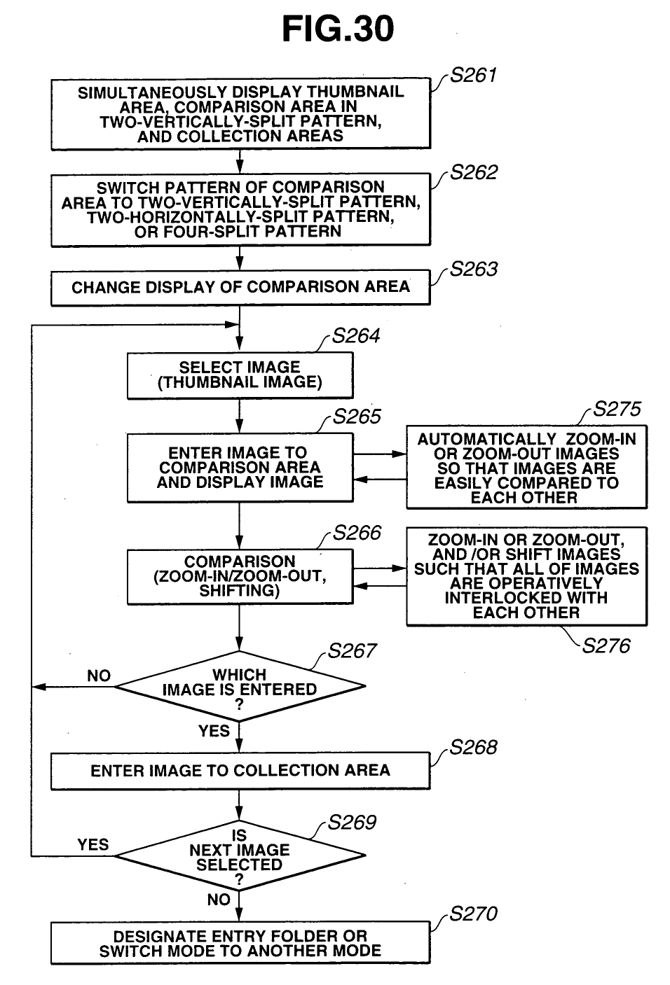

FIG. 30 is a flowchart showing the operation in the VLB mode, the operation being performed mainly through the comparative selection means 220.

When the mode shifts to the VLB mode, the thumbnail area 241, a comparison area 251 in a two-image-vertically-split pattern, and the collection areas 238A and 238B are simultaneously displayed as shown in FIGS. 31A to 33D (step S261). In this case, for example, the two-image-vertically-split pattern is set as an initial state obtained when the mode shifts to the VLB mode.

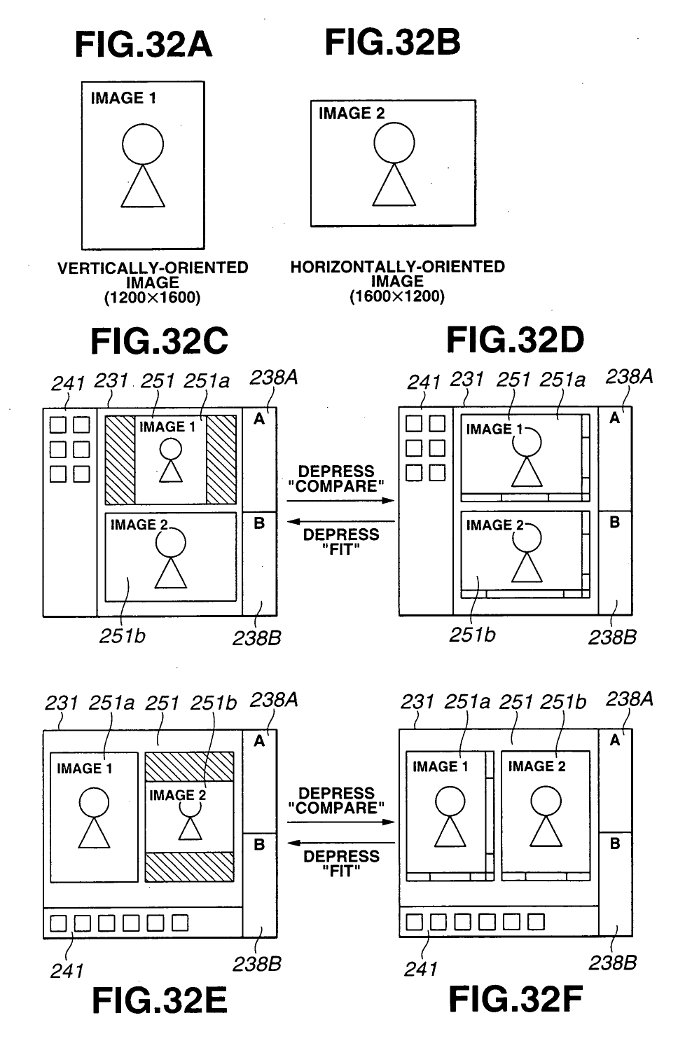

After that, when necessary, the user switches the pattern of the comparison area 251 to another pattern with a display-mode change button (S262). According to the third embodiment, the two-image-vertically-split pattern (refer to FIGS. 32C and 32D), a two-image-horizontally-split pattern (refer to FIGS. 32E and 32F), and a four-image-split pattern, namely, three kinds of patterns are preset as switchable patterns of the comparison area 251. The pattern is not limited to the above examples.

If the pattern of the comparison area 251 is switched to another pattern in step S262, the display is changed in accordance with the operation (step S263). If the pattern of the comparison area 251 is not switched, the process proceeds to step S264 without particularly processing in steps S262 and S263.

Subsequently, when necessary, the user selects any one of the thumbnail images displayed in the thumbnail area 241 using the mouse 212 by dragging the image and dropping it on the comparison area 251 (step S264).

The selected image is entered into the comparison area 251 and is displayed therein (step S265).

In this instance, when a compare button, which will be described later, is operated, the comparative display means 225 automatically enlarges or reduces a plurality of images displayed in the comparison area 251 to set each image to a proper size so that the images are easily compared to each other (step S275).

After the images are displayed in the comparison area 251 in step S265 mentioned above, the user compares the images. In this instance, when necessary, the user zooms out or zooms in an image in the comparison area 251 using a zoom-out button or a zoom-in button, and/or moves an image using a moving pointer, which is set through a moving-pointer set button. The displayed images in accordance with the operation are processed (step S266).

At this time, if an interlocking button is ON, the images displayed in the comparison area 251 are zoomed in or zoomed out, and/or moved such that they are operatively interlocked with each other (step S276). When the compare button is operated in step S275 mentioned above, the interlocking is similarly effective.

Subsequently, whether any of the images displayed. in the comparison area 251 is entered into the collection area 238A or 238B is determined (step S267). If NO, the process is returned to step S264, thus repeating the above-mentioned steps.

If the entry is performed in step S267, the image is entered into the collection area 238A or 238B according to the user's designation (step S268).

After that, whether the next image is to be selected is determined (step S269). If YES, the process is returned to step S264 mentioned above, thus repeating the above-mentioned steps.

On the other hand, if the next image is not selected, an entry folder is designated. Alternatively, the mode is changed to another mode (step S270).

Subsequently, the operation in steps S275 and 276 will now be described in detail with reference to FIGS. 31A to 33D.

FIGS. 31A to 31F are diagrams showing a process of easily making a comparison between images of which resolutions only are different. FIGS. 31A and 31B show respective original image data items. FIGS. 31C, 31D, 31E, and 31F show the images in the comparison area 251 in the VLB mode. The layout of the comparison area 251 denotes a two-horizontally-split pattern.

In the VLB mode, as shown in FIGS. 31C to 31F, the thumbnail area 241, the comparison area 251, and the collection areas 238A and 238B are displayed in a window 231. In the thumbnail area 241, images stored in the auxiliary storage device 215 are arranged and displayed as thumbnail images. In the comparison area 251, images selected from among the images in the thumbnail area 241 via dragging and dropping are displayed in order to make a comparison therebetween. In each of the collection areas 238A and 238B, an image selected from the images displayed in the comparison area 251 is entered, and entered images are stored as a group. In the case shown in FIGS. 31A to 31F, the layout of the comparison area 251 is set to a two- vertically-split pattern. That is, an image display space 251a is arranged in an upper portion of the comparison area 251 and an image display space 251b is arranged in a lower portion thereof. These image display spaces 251a and 251b are formed in the same size.

FIG. 31A shows a first image (image 1) of (1600 horizontal pixels)×(1200 vertical pixels). FIG. 31B shows a second image (image 2) of (800 horizontal pixels)×(600 vertical pixels). A comparison between the first and second images in the comparison area 251 in the VLB mode will now be described. These images are obtained by shooting the same subject with different resolutions. Although the number of dots constituting the first image differs from the number of dots constituting the second image, the picture composition of the first image is the same as that of the second image. Images having only different resolutions (numbers of pixels constituting the respective images) can be obtained as follows: One of two images, obtained by shooting in the same horizontal orientation at the same zoom magnification (zoom magnification of a photographing optical system upon shooting), is subjected to pixel skipping and the resultant image is stored. Pixel skipping is set by, for example, changing a shooting mode related to the quality (definition) of an image.

FIG. 31C shows an initial state obtained when the image shown in FIG. 3 1A is dragged from the thumbnail area 241 and dropped on the image display space 251a via dragging and dropping or the like and the image displayed shown in FIG. 31B is similarly displayed in the image display space 251b.

In this initial state in which the respective images are displayed in the image display spaces 251a and 251b, as shown in FIG. 31C, the images are displayed at lx magnification (100% display). In other words, the images are displayed such that the number of vertical and horizontal dots constituting each image matches the number of dots of the corresponding image displayed in the display device 216. In this case, the number of pixels in each of the image display spaces 251a and 251b is set to, for example, (400 horizontal dots)×(300 vertical dots). Accordingly, only the central portion, comprising 400×300 dots, of each of the images in FIGS. 31A and 31B is displayed in the image display space. At this time, the resolution of the image in FIG. 31A is different from that in FIG. 31B, so that the number of pixels constituting the main subject of the image in FIG. 31A is different from that in FIG. 31B. Thus, the display size of the main subject in the image display space 251a is different from that in the image display space 251b.

In the ON state of the interlocking button, when the user zooms in the images in FIG. 31C at, for example, 2× magnification using the zoom-in button, the respective images displayed in the image display spaces 251a and 251b are zoomed in at 2× magnification such that the images are operatively interlocked with each other, and the resultant images are displayed. At this time, the display size of the main subject in the upper space is different from that in the lower space in FIG. 31C. Accordingly, the display size of the main subject in the upper space is also different from that in the lower space in FIG. 31E.

When the interlocking button is OFF, only the image in the image display space 251a or 251b which is active is zoomed in. In the ON state of the interlocking button, when the user operates the zoom-out button, the images are similarly zoomed out such that they are operatively interlocked with each other. When the user performs the moving operation, the images are moved such that they are similarly operatively interlocked with each other.

When the compare button is operated in FIG. 31C, display magnification rate is changed so that a comparison between the images can be easily made. In this case, the image of 1600×1200 dots shown in FIG. 31A is zoomed out by 25% (¼) such that the image is fitted into the image display space 251a of 400×300 dots. The image of 800×600 dots shown in FIG. 31B is zoomed out by 50% (½) such that the image is fitted into the image display space 251b of 400×300 dots. Thus, the respective main subjects have the same-sized images, so that the comparison between the images can be easily made.

As for the images of which resolutions only are different as shown in FIGS. 31A and 31B, when the user operates a fit button in place of the compare button, the similar effect can be produced. The fit button is used to display an image in an image display space as large as possible such that the image has no part which lies off-screen.

Further, in the ON state of the interlocking button, when the user zooms in the images in FIG. 31D at, for example, 2× magnification using the zoom-in button, the respective images in the image display spaces 251a and 251b are zoomed in at 2× magnification such that they are operatively interlocked with each other, and the resultant images are displayed. At this time, the display size of the main subject in the image display space 251a is the same as that in the image display space 251b in FIG. 31D. Accordingly, the display size of the main subject in the image display space 251a is the same as that in the image display space 251b upon zoom-in. In the zoom-in at 2× magnification, the image shown in FIG. 31A is zoomed out by 50% (½) and is then displayed. The image shown in FIG. 31B is displayed at 1× magnification (100%). Further, the respective images are zoomed out and/or moved such that the images are operatively interlocked with each other in a manner similar to the above description.

FIGS. 32A to 32F are diagrams showing a process of easily making a comparison between images of which resolutions only are different. FIGS. 32A and 32B show original image data items. FIGS. 32C and 32D show a case where the layout of the comparison area 251 is set to a two-vertically-split pattern. FIGS. 32E and 32F show a case where the layout of the comparison area 251 is set to a two-horizontally-split pattern.

FIG. 32A shows a first image (image 1) in which the number of horizontal and vertical pixels is 1200×1600 dots, the image being captured by shooting in a vertical orientation. FIG. 32B shows a second image (image 2) in which the number of horizontal and vertical pixels is 1600×1200 dots, the image being captured by shooting in a horizontal orientation. A comparison therebetween in the comparison area 251 in the VLB mode will now be described. These images are captured by shooting the same subject with the same resolution at the same zoom magnification. Only the vertical and horizontal orientations of the images are different from each other.

In an initial state in which the respective images shown in FIGS. 32A and 32B are read, the images are displayed at 1× magnification as mentioned above. Since the image display spaces 251a and 251b each comprise 400×300 dots, the whole of the image comprising 1200×1600 dots in FIG. 32A is not displayed and the whole of the image comprising 1600×1200 dots in FIG. 32B is not displayed.

In this initial state, when the user depresses, for example, the fit button, as shown in FIG. 32C, the whole of the image is displayed in each of the image display spaces 251a and 251b such that the image has no part which lies off-screen. Since the image in FIG. 32A is vertically oriented, blank portions (where any image is not displayed) are arranged on both the sides in the image display space 251a in the fit display.

More specifically, the image in FIG. 32A is zoomed out by 18.75% (3/16) such that 1600 vertical dots match 300 vertical dots constituting the image display space 251a and the resultant image is displayed. The image in FIG. 32B is zoomed out by 25% (¼) such that (1600×1200) dots match (400×300) dots constituting the image display space 251b and the resultant image is displayed.

In the state shown in FIG. 32C, when the user operates the compare button, the display magnifications are changed as shown in FIG. 32D such that a comparison between the images is easily made. In this instance, the image in FIG. 32A is zoomed out by about 33% (⅓) so that the image with 1200 horizontal dots is fitted into the image display space 251a with 400 horizontal dots without having no blank portions in the image display space 251a, and the resultant image is displayed. The image in FIG. 32B is also zoomed out by about 33% (⅓) in accordance with the zoom-out of the image in FIG. 32A. The zoom-out in this case is performed to the original image data items shown in FIGS. 32A and 32B. As compared with the all images in FIG. 32C, the all images in FIG. 32D are zoomed in.

In the state shown in FIG. 32D, when the user operates the fit button, the display mode again shifts to that in FIG. 32C.

Further, when the interlocking button is ON, in the state shown in each of FIGS. 32C and 32D, the images are zoomed in or zoomed out, and/or moved such that they are operatively interlocked with each other in a manner similar to the case shown in FIGS. 31A to 31F.

On the other hand, in the case where the layout of the comparison area 251 is set to a two-image-horizontally-split pattern, when the fit button is depressed after the initial state, as shown in FIG. 32E, blank portions are formed in the image display space 251b where the horizontally-oriented image is displayed. In this instance, it is assumed that the image display spaces 251a and 251b each comprise 300×400 dots as the number of pixels in the horizontal and vertical directions.

In this case, the image shown in FIG. 32A is zoomed out by 25% (¼) such that 1200×1600 dots match 300×400 dots constituting the image display space 251b, and the resultant image is displayed. The image shown in FIG. 32B is zoomed out by 18.75% (3/16) such that 1600 horizontal dots match 300 horizontal dots constituting the image display space 251a, and the resultant image is displayed.

In the state shown in FIG. 32E, when the compare button is operated, as shown in FIG. 32F, the display magnifications are changed such that a comparison between the images is easily made. In this case, the image shown in FIG. 32B is zoomed out by about 33% (⅓) so that the image with 1200 vertical dots is fitted into the image display space 251b with 400 vertical dots without having no blank portions in the image display space 251b, and the resultant image is displayed. The image shown in FIG. 32B is also zoomed out by about 33% (⅓) in accordance with the zoom-out of the image shown in FIG. 32A. The zoom-out in this case is performed on the original image data items shown in FIGS. 32A and 32B.

As mentioned above, as for the images which are different from each other in term of the horizontal and vertical orientations, when the compare button is operated, the respective images can have the same-sized main subjects. As a result, the comparison therebetween can be easily made.

When the interlocking button is ON, in the state shown in each of FIGS. 32E and 32F, the respective images are zoomed in or zoomed out, and/or moved such that the images are operatively interlocked with each other in a manner similar to the above description.

In this case, when the compare button is operated, the magnification factors are adjusted such that the main subject displayed in the image display space 251a is the same as that in the image display space 251b. When the adjustment is controlled such that the main subject displayed in the image display space 251a is substantially the same in the image display space 251b, the process can be simplified.

For example, referring to FIG. 32D, the image in the image display space 251b may be displayed as an image zoomed out by 25% similar to that shown in FIG. 32C. And, only the image in the image display space 251a may be changed such that the image is zoomed out by about 33%. Alternatively, referring to FIG. 32F, the image in the image display space 251a may be displayed as an image zoomed out by 25% similar to that shown in FIG. 32E. And, only the image in the image display space 251b may be changed such that the image is zoomed out by about 33%.

Under condition that the aspect ratio of each image is 3:4, namely, relatively approximate to a square, if the above-mentioned simplified process is performed, ease of comparison between the images is not deteriorated. On the other hand, when the aspect ratio is remarkably different from a square, for example, an image is captured by shooting in a panoramic mode, it is desirable to display images at the same magnification so as to have no blank portions as mentioned above.

In the above description, only the vertical and horizontal orientations have been considered as the orientations of images. In actual shooting, a shooting person may capture an image by shooting while inclining a camera. As for such an oblique image, the image can be obliquely cut out such that a main subject is arranged in a normal position.

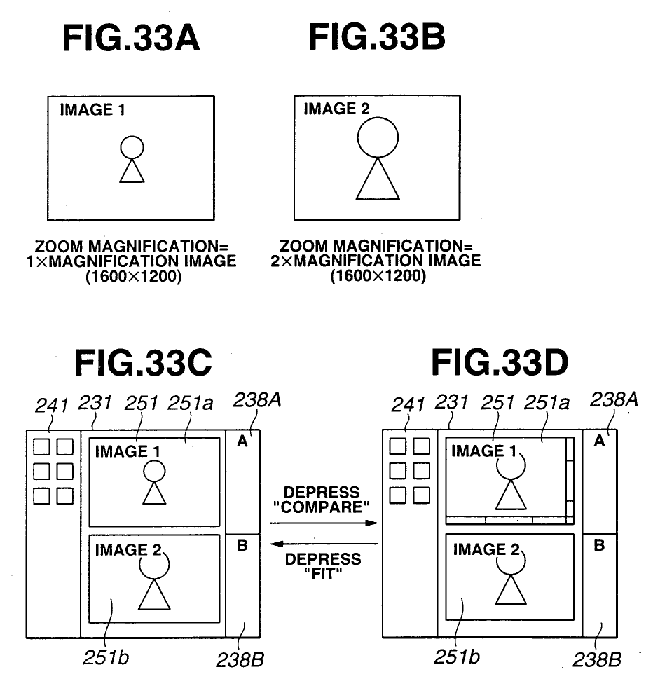

FIGS. 33A to 33D are diagrams showing a process of easily making a comparison between images of which only zoom magnifications are different.

FIG. 33A shows a first image (image 1) in which the number of horizontal and vertical pixels is 1600×1200 dots, the first image being captured by shooting in a horizontal orientation such that the zoom magnification is set to, for example, an appropriate wide-angle magnification. FIG. 33B shows a second image (image 2) in which the number of horizontal and vertical pixels is 1600×1200 dots, the second image being captured by shooting in a horizontal orientation such that the zoom magnification is, for example, two times that in FIG. 33A. A comparison therebetween in the comparison area 251 in the VLB mode will now be described. These images are captured by shooting the same subject with the same resolution in the same horizontal orientation. Only the zoom magnifications of the images are different from each other.

In an initial state in which the images shown in FIGS. 33A and 33B are read, as mentioned above, the images are displayed at 1× magnification. Since the image display spaces 251a and 251b each comprise 400×300 dots, the whole image comprising 1600×1200 dots, shown in each of FIGS. 33A and 33B, is not displayed.

In this initial state, for example, if the fit button is depressed, as shown in FIG. 33C, the whole image is displayed in each of the image display spaces 251a and 251b without having no part lying off-screen.

Referring to FIG. 33C, more specifically, the respective images, each comprising (1600 horizontal dots)×(1200 vertical dots), shown in FIG. 33A and 33B are zoomed out by 25% (¼) so that the images are fitted into the respective image display spaces 251a and 251b, each comprising (400 horizontal dots)×(300 vertical dots). The resultant images are displayed.

When the compare button is operated on the condition shown in FIG. 33C, as shown in FIG. 33D, display magnifications are changed such that the comparison between the images is easily made. In the process, information of the zoom magnifications of the respective images is first obtained. The information is added upon capturing the corresponding image as header information in a file in which the image is stored. On the basis of the obtained information items, it is recognized that the image shown in FIG. 33B is obtained at the zoom magnification that is two times that of the image shown in FIG. 33A. Then, the image with smaller zoom magnification in FIG. 33A is zoomed in. In this case, the display image in FIG. 33C is zoomed in. As compared with the original image data items shown in FIGS. 33A and 33B, the images shown in FIG. 33D are zoomed out.

In other words, the image displayed in the image display space 251b is held as an image zoomed out by 25% (¼). In the image display space 251a, the image zoomed out by 25% (¼) is enlarged to the image zoomed out by 50% (½).

Further, when the interlocking button is ON, in each of the conditions shown in FIGS. 33C and 33D, the images are zoomed in or zoomed out, and/or moved such that they are operatively interlocked with each other in a manner similar to the above description.

Consequently, the respective images have the same-sized main subjects. Thus, the comparison therebetween can be easily made.

More generally, a plurality of images displayed in the comparison area 251 may be different from each other in terms of any two or more of the resolution, the orientation, and the zoom magnification. In this case, the above-described processes are used in combination. Thus, the processes such as zoom-in and zoom-out are performed such that a comparison between the images can be easily made by merely operating the compare button.

When three or more images are displayed in the comparison area 251, the processes are performed such that the respective images have the same-sized (or substantially the same-sized) main subjects.

Further, when the above-mentioned image processing program is recorded in a computer-readable recording medium and another computer reads and runs the program, the program can permit the computer to similarly function.

In the above description, the image processing program is run on the general purpose PC connected to a digital camera, thus realizing the image processing apparatus. The invention is not limited to the above arrangement. A dedicated image processing apparatus may be constructed such that the same functions as those of a computer, on which the image processing program runs, are realized by hardware.

According to the third embodiment, in a comparison between several different images with various resolutions and various orientations at various zoom magnifications, when the compare button is merely operated, main subjects of the respective images can be displayed in substantially the same size with operationality. Thus, the comparison between the images can be easily made.

In this instance, if the interlocking button is ON, the images are zoomed in or zoomed out, and/or moved such that they are operatively interlocked with each other. Thus, the operationality is further improved.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An image processing apparatus comprising:
first display means for displaying one or more images of a plurality of images;
classification means for putting each image displayed through the first display means into any one of a plurality of categories;
designation means for designating one or more categories among the categories;
second display means for displaying one or more images each of which belongs to any of the one or more categories designated by the designation means; and
selective designation means for selecting and designating an image from among the images displayed through the second display means.

2. The apparatus according to claim 1, wherein the first display means includes any one of browse display means for displaying a plurality of reduced images in an array, view display means for displaying one single image, and virtual-light-box display means for displaying two or more images to compare the images.

3. The apparatus according to claim 1, wherein the second display means includes any one of browse display means for displaying a plurality of reduced images in an array, view display means for displaying one single image, and virtual-light-box display means for displaying two or more images to compare the images.

4. The apparatus according to claim 1, further comprising:
storage control means for grouping one or more images selected and designated through the selective designation means and storing the grouped images into the same area in a recording medium.

5. The apparatus according to claim 1, wherein the selective designation means comprises further means for selecting and designating an image from among the images displayed through the first display means.

6. An image processing program comprising:
a first display procedure for displaying one or more images of a plurality of images;
a classification procedure for putting each image, displayed in the first display procedure, into any one of a plurality of categories;
a designation procedure for designating one or more categories from among the categories;
a second display procedure for displaying one or more images each of which belongs to any of the one or more categories designated in the designation procedure; and
a selective determination procedure for selecting and designating an image from among the images displayed in the second display procedure.

7. A recording medium used for computer-readably recording an image processing program, the image processing program comprising:
a first display procedure for displaying one or more images of a plurality of images;
a classification procedure for putting each image, displayed in the first display procedure, into any one of a plurality of categories;
a designation procedure for designating one or more categories from among the categories;
a second display procedure for displaying one or more images each of which belongs to any of the one or more categories designated in the designation procedure; and
a selective designation procedure for selecting and designating an image from among the images displayed in the second display procedure.

8. An image processing method comprising:
a first display procedure for displaying one or more images of a plurality of images;
a classification procedure for putting each image, displayed in the first display procedure, into any one of a plurality of categories;
a designation procedure for designating one or more categories from among the categories;
a second display procedure for displaying one or more images each of which belongs to any of the one or more categories designated in the designation procedure; and
a selective designation procedure for selecting and designating an image from among the images displayed in the second display procedure.

9. An image processing apparatus comprising:
display means for displaying first, second, and third display areas in a common screen, the first display area being used to display a plurality of reduced images arranged in a predetermined first order, the second display area including two or more image display spaces arranged in a predetermined second order, one of the image display spaces being movably set as a target image display space, the second display area being used to compare a plurality of images, the third display area being used to store an image selected from the images displayed in the second display area and display the selected image as a reduced image;
selection means for selecting one reduced image from among the reduced images displayed in the first display area;
display control means for controlling the display means to display an image, corresponding to the reduced image selected through the selection means, in the target image display space in the second display area;
display holding means for releasably holding a display state of the image displayed in the target image display space in the second display area;
target image-space shifting means for automatically moving the target image display space by one in accordance with the second order when the display holding means holds the image; and
selective designation means for selecting and designating the image displayed in the second display area to store the image into the third display area.

10. The apparatus according to claim 9, further comprising:
collective-display set means for setting a collective display, wherein
while the collective display is set through the collective-display set means, when one reduced image is selected through the selection means, the display control means displays an image corresponding to the selected reduced image in the target image display space in the second display area, and also displays, in accordance with the second order, respective images corresponding to other reduced images that follow the reduced image selected in accordance with the first order, in the predetermined number of image display spaces excluding the image display space where the image is held by the display holding means.

11. The apparatus according to claim 9, wherein the selection means includes means for selecting any one of the reduced images arranged and displayed in the first display area with a single-click.

12. The apparatus according to claim 9, wherein the selection means includes means for moving any one of the reduced images arranged and displayed in the first display area to any of the image display space in the second display area with drag- and-drop.

13. The apparatus according to claim 9, wherein the selection means includes an image add button and one reduced image is selected from among the reduced images displayed in the first display area using the image add button.

14. An image processing program comprising:
a display procedure for displaying first, second, and third display areas in the same screen, the first display area being used to display a plurality of reduced images arranged in a predetermined first order, the second display area including two or more predetermined number of image display spaces arranged in a predetermined second order, one of the image display spaces being movably set as a target image display space, the second display area being used to compare a plurality of images, the third display area being used to store an image selected from the images displayed in the second display area and display the selected image as a reduced image;
a selection procedure for selecting one reduced image from among the reduced images displayed in the first display area;
a display control procedure for controlling an image corresponding to the reduced image selected in the selection procedure so that the image is displayed in the target image display space of the second display area in the display procedure;
a display holding procedure for releasably holding the display state of the image displayed in the target image display space in the second display area;
a target image-space shifting procedure for automatically moving the target image display space by one in accordance with the second order when the image is held in the display holding procedure; and
a selective designation procedure for selecting and designating the image displayed in the second display area to store the image into the third display area.

15. A recording medium used for computer-readably recording an image processing program, the image processing program comprising:
a display procedure for displaying first, second, and third display areas in the same screen, the first display area being used to display a plurality of reduced images arranged in a predetermined first order, the second display area including two or more predetermined number of image display spaces arranged in a predetermined second order, one of the image display spaces being movably set as a target image display space, the second display area being used to compare a plurality of images, the third display area being used to store an image selected from the images displayed in the second display area and display the selected image as a reduced image;
a selection procedure for selecting one reduced image from among the reduced images displayed in the first display area;
a display control procedure for controlling an image corresponding to the reduced image selected in the selection procedure so that the image is displayed in the target image display space of the second display area in the display procedure;
a display holding procedure for releasably holding the display state of the image displayed in the target image display space in the second display area;
a target-image-space moving procedure for automatically moving the target image display space by one in accordance with the second order when the image is held in the display holding procedure; and
a selective designation procedure for selecting and designating the image displayed in the second display area to store the image into the third display area.

16. An image processing method comprising:
a display procedure for displaying first, second, and third display areas in the same screen, the first display area being used to display a plurality of reduced images arranged in a predetermined first order, the second display area including two or more predetermined number of image display spaces arranged in a predetermined second order, one of the image display spaces being movably set as a target image display space, the second display area being used to compare a plurality of images, the third display area being used to store an image selected from the images displayed in the second display area and display the selected image as a reduced image;
a selection procedure for selecting one reduced image from among the reduced images displayed in the first display area;
a display control procedure for controlling an image corresponding to the reduced image selected in the selection procedure so that the image is displayed in the target image display space of the second display area;
a display holding procedure for releasably holding the display state of the image displayed in the target image display space in the second display area;
a target-image-space moving procedure for automatically moving the target image display space by one in accordance with the second order when the image is held in the display holding procedure; and
a selective designation procedure for selecting and designating the image displayed in the second display area to store the image into the third display area.

17. An image processing apparatus comprising:
display control means for controlling a plurality of different images such that the images are displayed in respective same-sized image display areas on a common screen;
comparison-image-size adjusting means for enlarging or reducing the display size of at least one of the images such that subjects included in the respective images displayed in the image display areas are displayed in substantially the same size on the screen, the subjects indicating the same subject;
image processing means for performing image processing on any one or more images of the images displayed in the image display areas; and
interlocking means for allowing the image processing means to perform image processing on all of the images displayed in the image display areas such that the images are operatively interlocked with each other.

18. The apparatus according to claim 17, wherein
the images are different from each other in terms of the number of pixels constituting each image, and
the comparison-image-size adjusting means enlarges or reduces the display size of each image based on information related to the number of pixels of the image.

19. The apparatus according to claim 17, wherein
the images are different from each other in terms of the orientation of each image, and
the comparison-image-size adjusting means enlarges or reduces the display size of each image based on information related to the orientation of the image.

20. The apparatus according to claim 17, wherein
the images are different from each other in terms of the zoom magnification of a photographing optical system upon capturing each image by shooting, and
the comparison-image-size adjusting means enlarges or reduces the display size of each image based on information related to the zoom magnification of the photographing optical system upon capturing the image by shooting.

21. The apparatus according to claim 17, wherein the image processing means includes display scaling changing means for zooming in or zooming out a displayed image within the corresponding image display area.

22. The apparatus according to claim 17, wherein the image processing means includes display image moving means for moving a displayed image within the corresponding image display area.

23. An image processing program comprising:
a display control procedure for controlling a plurality of different images such that the images are displayed in respective same-sized image display areas on a common screen;
a comparison-image-size adjusting procedure for enlarging or reducing the display size of at least one of the images such that subjects included in the respective images displayed in the image display areas are displayed in substantially the same size on the screen, the subjects indicating the same subject;
an image processing procedure for performing image processing on any one or more images of the images displayed in the image display areas; and
an interlocking procedure for performing the image processing in the image processing procedure to all of the images displayed in the image display areas such that the images are operatively interlocked with each other.

24. A recording medium used for computer-readably recording an image processing program, the image processing program comprising:
a display control procedure for controlling a plurality of different images such that the images are displayed in respective same-sized image display areas on a common screen;
a comparison-image-size adjusting procedure for enlarging or reducing the display size of at least one of the images such that subjects included in the respective images displayed in the image display areas are displayed in substantially the same size on the screen, the subjects indicating the same subject;
an image processing procedure for performing image processing on any one or more images of the images displayed in the image display areas; and
an interlocking procedure for performing the image processing in the image processing procedure to all of the images displayed in the image display areas such that the images are operatively interlocked with each other.

25. An image processing method comprising:
a display control procedure for controlling a plurality of different images such that the images are displayed in respective same-sized image display areas on a common screen;
a comparison-image-size adjusting procedure for enlarging or reducing the display size of at least one of the images such that subjects included in the respective images displayed in the image display areas are displayed in substantially the same size on the screen, the subjects indicating the same subject;
an image processing procedure for performing image processing on any one or more images of the images displayed in the image display areas; and
an interlocking procedure for performing the image processing in the image processing procedure to all of the images displayed in the image display areas such that the images are operatively interlocked with each other.
